(19) United States
(12) Patent Application Publication
(10) Pub. No.: US 2026/0115334 A1
(43) Pub. Date: Apr. 30, 2026

(54) CONJUGATES, COMPOSITIONS, AND METHODS FOR HYDROXYAPATITE-TARGETED IMAGING AND THERAPY

(71) Applicant: PURDUE RESEARCH FOUNDATION, West Lafayette, IN (US)

(72) Inventors: Philip Stewart LOW, West Lafayette, IN (US); Losha Dasol JUNG, West Lafayette, IN (US); Madduri SRINIVASARAO, West Lafayette, IN (US); Stewart LOW, West Lafayette, IN (US); Jeffery NIELSEN, West Lafayette, IN (US)

(21) Appl. No.: 19/433,382

(22) Filed: Dec. 26, 2025

Related U.S. Application Data
(63) Continuation-in-part of application No. 18/814,372, filed on Aug. 23, 2024.
Application No. 18/814,372 is a continuation-in-part of application No. PCT/US2023/013863, filed on Feb. 24, 2023.
(60) Provisional application No. 63/313,395, filed on Feb. 24, 2022.

(51) Int. Cl. A61K 51/08; A61K 45/06

(52) U.S. Cl. CPC A61K 51/088; A61K 45/06; A61K 2121/00; A61K 2123/00

(57) ABSTRACT

Conjugates that target hydroxyapatite, compositions comprising the same, and methods of use of such conjugates and compositions for radio-imaging or radiotherapy.

### PRIORITY

This patent application is related to, claims the priority benefit of, and is a continuation-in-part of U.S. patent application Ser. No. 18/814,372, filed Aug. 23, 2024, which is related to, claims the priority benefit of, and is a continuation-in-part of International Patent Application No. PCT/US2023/013863, filed Feb. 24, 2023, which is related to and claims the priority benefit of U.S. Provisional Patent Application No. 63/313,395 filed Feb. 24, 2022. The contents of the foregoing applications are hereby incorporated by reference in their entireties into this disclosure.

### TECHNICAL FIELD

The present disclosure relates to conjugates that target hydroxyapatite, compositions comprising the same, and methods of use for such conjugates and compositions for imaging and therapy.

### BACKGROUND

It is difficult to deliver drugs to bone because its composition differs from other organs. It is mostly composed of calcium phosphate crystals known as hydroxyapatite. Current systemic treatment can be thus highly inefficient for various bone diseases because the majority of the drug is captured by the visceral organs and ultimately excreted via urine, instead of concentrating at the diseased and damaged site. Increasing the dosage may improve the onsite localization but can also increase off-target systemic side effects. Accordingly, effective bone-targeting systems are needed to improve the benefits of drugs on bone diseases.

Among various bone diseases, bone cancers (such as bone metastases due to breast or prostate cancer, for example) have especially high mortality rates as current systemic chemotherapies and radiotherapies are ineffective against eradicating or reducing tumor growth. At most, these treatments provide relief from pain induced by tumor-induced bone lesions and, consequently, many bone cancers are widely accepted as incurable. Therefore, the development of bone-targeted therapy is highly urgent to improve the quality of life and survival rate of patients.

### SUMMARY

Provided is a conjugate of Formula I:

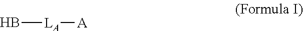

or a pharmaceutically acceptable salt thereof, wherein:
HB is a radical of a (e.g., straight chain or branched-chain) a D-γ-carboxyglutamic acid ligand, L-γ-carboxyglutamic acid ligand, or a mixture of the foregoing or derivatives thereof that binds hydroxyapatite (e.g., a biological targeting moiety);
A is a radio-imaging agent, a radio-sensitizing agent, a radio-protecting agent, or a radiotherapeutic agent; and
L_{A} is a linker that binds an HB and A or absent.

HB can comprise a radical of a straight-chain or branched chain D-γ-carboxyglutamic acid ligand, L-γ-carboxyglutamic acid ligand, or a mixture of the foregoing. In certain embodiments, HB comprises an amino acid or derivative thereof and at least ⅓ of the amino acid of HB is acidic in nature. HB can comprise 1 to 100 D-γ-carboxyglutamic acid residues, L-γ-carboxyglutamic acid residues, or a mixture of the foregoing or derivatives thereof. HB can comprise or have 7-20 D-γ-carboxyglutamic acid residues. In certain embodiments, HB is 7 D-γ-carboxyglutamic acid residues. HB can comprise or have 7-20 L-γ-carboxyglutamic acid residues. In certain embodiments, HB is 7 L-γ-carboxyglutamic acid residues.

In certain embodiments, provided is a conjugate of Formula I:

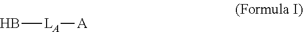

or a pharmaceutically acceptable salt thereof, wherein:
HB is a radical of a (e.g., straight chain or branched-chain) polyanionic, polyacidic, and/or polyelectrolytic ligand that binds hydroxyapatite (e.g., a biological targeting moiety);
A is a radio-imaging agent, a radio-sensitizing agent, a radio-protecting agent, or a radiotherapeutic agent; and
L_{A} is a linker that binds an HB and A or absent.

HB can comprise a radical of a straight chain polyanionic, polyacidic, and/or polyelectrolytic ligand that binds hydroxyapatite. HB can comprise an amino acid or a derivative thereof. HB can comprise L-aspartic acid, D-aspartic acid, L-glutamic acid, D-glutamic acid, D-γ-carboxyglutamic acid, L-γ-carboxyglutamic acid, or a mixture of two or more of the foregoing or derivatives thereof.

In some embodiments, HB can further comprise (i) a serum albumin binder (AB) conjugated directly to HB, or (ii) AB and a linker L_{AB} (AB-L_{AB}), wherein AB-L_{AB} is conjugated to HB via L_{AB}. In some embodiments, one or more peptide bonds of HB and/or A are arranged in a relative cis orientation. In some embodiments, one or more peptide bonds of HB and/or A are arranged in a relative trans orientation.

In some embodiments, L_{A}, L_{AB}, or both L_{A} and L_{AB} is/are in an L-configuration. In some embodiments, L_{A}, L_{AB}, or both L_{A} and L_{AB} is/are in a D-configuration AB can be attached to an amine or carboxyl end of polyacidic peptide or an active group, such as carboxyl, amine, or thiol groups, of repeating anionic, acidic, or electrolytic moieties of HB.

HB can comprise a radical of a straight-chain polyanion of formula X-A₁, X-A₂, X-A₃, or X-A₄ as follows:

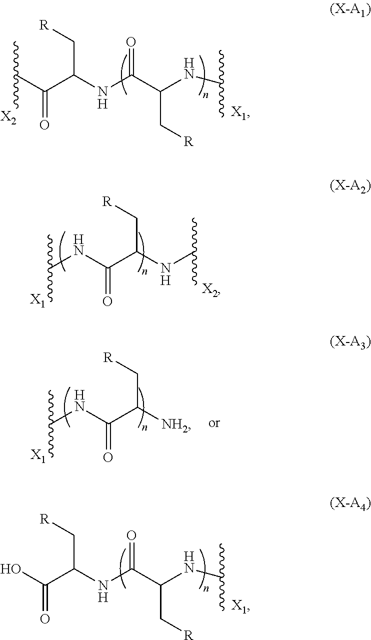

wherein:
R is COOH for L- or D-aspartic acid, CH₂COOH for L- or D-glutamic acid, HOOC—CH₂—CH(COOH)—CH(NH₂)—COOH or D- or L-γ-carboxyglutamic acid, or CH₂CH₂COOH for an unnatural, acidic amino acid;
n=1-50;

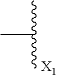

represents the point of attachment to L_{A}; and,
when HB further comprises AB or AB-L_{AB},

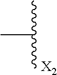

represents the point of attachment to AB or L_{AB}.

In some embodiments, HB may not (e.g., does not) comprise an albumin binder, as shown in formulae X-A₃ and X-A₄. HB can comprise a radical of a branched-chain polyanion of formula X-A₅, X-A₆, X-A₇, or X-A₈ as follows:

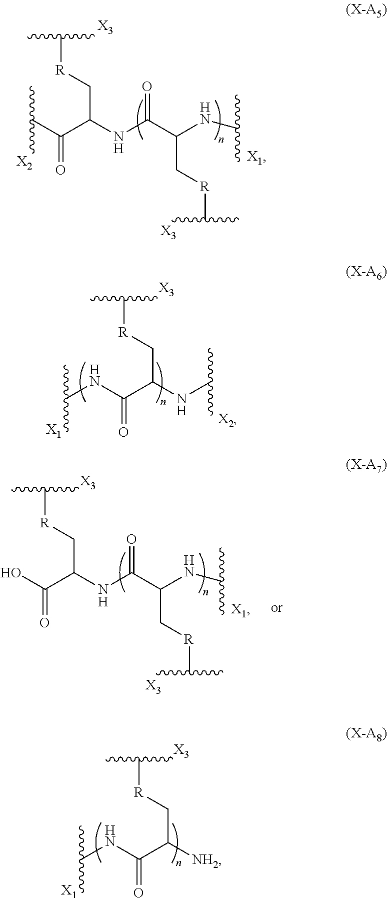

wherein:
R is COO— for L- or D-aspartic acid, CH₂COO— for L- or D-glutamic acid, HOOC—CH₂—CH(COOH)—CH(NH₂)—COOH or D- or L-γ-carboxyglutamic acid, or CH₂CH₂COO— for an unnatural, acidic amino acid;
n=1-50;

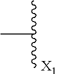

represents the point of attachment to L_{A};
when HB further comprises AB or AB-L_{AB},

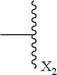

represents the point of attachment to AB or L_{AB}; and

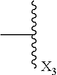

represents the point of attachment to hydrogen or an HB. In some embodiments, HB may not comprise an albumin binder, such as shown in formulae X-A₇ and X-A₈.

L_{A} can comprise one or more amino acids. L_{A} can comprise a brush border membrane (BBM) linker. L_{A} can comprise a BBM linker comprising Met-Val-Lys. L_{A} can comprise a BBM linker comprising Gly-Tyr-Lys.

Either or both of L_{A} and L_{AB} can comprise a slow-release linker. L_{A} can comprise a slow-release linker. L_{A} can comprise a quick-release linker. L_{A} can comprise L₁ or L₂ as follows:

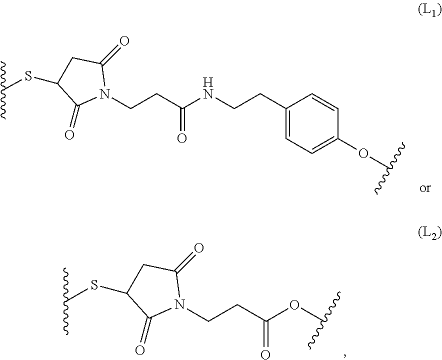 [SMILES: *Oc1ccc(CCNC(=O)CCN2C(=O)CC(S*)C2=O)cc1 ; *OC(=O)CCN1C(=O)CC(S*)C1=O]

wherein

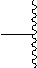

represents the point of attachment between L_{A} and A or L_{A} and HB.

Either or both of L_{A} and L_{AB} can comprise(s) a spacer. L_{A} and L_{AB} can be conjugated to the N- or C-terminus of HB, any monomer of a polymer thereof, or any substituent of any monomer of a polymer thereof.

A can comprise a chelator. A can comprise DOTA (1,4,7,10-tetraazacyclododecane-1,4,7,10-tetraacetic acid) or a derivative thereof. A can be selected from the group consisting of DOTA or a derivative thereof; TETA (1,4,8,11-tetraazacyclotetradecane-1,4,8,11-tetraacetic acid) or a derivative thereof, HEHA (1,2,7,10,13-hexaazacyclooctadecane-1,4,7,10,13,16-hexaacetic acid) or a derivative thereof, PEPA (1,4,7,10,13-pentaazacyclopentadecane-N,N′,N″,N′″,N″″-pentaacetic acid); SarAr (1-N-(4-Aminobenzyl)-3,6,10,13,16,19-hexaazabicyclo[6.6.6]-eicosane-1,8-diamine) or a derivative thereof, NOTA (1,4,7-triazacyclononane-1,4,7-triacetic acid) or a derivative thereof, NETA (4-[2-(bis-carboxymethylamino)-ethyl]-7-carboxymethyl-[1,4,7]triazonan-1-yl) acetic acid or a derivative thereof, TRAP (1,4,7-triazacyclononane-1,4,7-tris[methyl(2-carboxyethyl)phosphinic acid) or a derivative thereof, HBED (N,N0-bis(2-hydroxybenzyl)-ethylenediamine-N,N0-diacetic acid) or a derivative thereof, 2,3-HOPO (3-hydroxypyridin-2-one) or a derivative thereof, PCTA (3,6,9,15-tetraazabicyclo[9.3.1]-pentadeca-1(15),11,13-triene-3,6,9-triacetic acid) or a derivative thereof; PDTA (1,3-propylenediaminetetraacetate) or a derivative thereof; NTA (nitrilotriacetate) or a derivative thereof, EDDS (ethylenediaminedisuccinate) or a derivative thereof; EDTA (ethylene diamine tetraacetic acid) or a derivative thereof; MGDA (N-(1-carboxylatoethyl)iminodiacetate) or a derivative thereof, DFO (desferrioxamine) or a derivative thereof, DTPA (diethylenetriaminepentaacetic acid) or a derivative thereof; CDTA ((1,2-cyclohexylenedinitrilo)tetraacetic acid) or a derivative thereof; CPTA (1,4,8,11-tetraazacyclotetradecane derivative) or a derivative thereof; OCTAPA (N,N0-bis(6-carboxy-2-pyridylmethyl)-ethylenediamine-N,N0-diacetic acid) or a derivative thereof, H2-MACROPA (N,N′-bis[(6-carboxy-2-pyridipmethyl]-4,13-diaza-18-crown-6) or a derivative thereof; H₂dedpa (1,2-[[carboxy)-pyridin-2-yl]-methylamino]ethane or a derivative thereof, HEDP (hydroxyethylidene diphosphonate or etideronate) or a derivative thereof, HEBD (N,N′-bis(2-hydroxybenzyl) ethylenediamine-N,N′-diacetic acid) or a derivative thereof, HYNIC (6-Hydrazinopridine-3-carboxylic acid) or a derivative thereof; DMSA (meso-2,3-dimercaptosuccinic acid) or a derivative thereof, and EC20-head comprising β-1-diaminopropionic acid, Asp, and Cys.

The chelator can chelate an α-emitting radioisotope, such as ²²⁵Ac. The chelator can chelate a β-emitting radioisotope, such as ¹⁷⁷Lu or ⁹⁰Y. The chelator can chelate a γ-emitting radioisotope, such as ¹¹¹In or ⁶⁷Ga. The chelator can chelate ¹⁸F, ⁴⁴Sc, ⁴⁷Sc, ⁵²Mn, ⁵⁵Co, ⁶⁴Cu, ⁶⁷Cu, ⁶⁷Ga ⁶⁸Ga, ⁸⁶Y, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁷⁷Lu, ¹⁸⁶Re, ¹⁸⁸Re, ²¹²Pb ²¹²Bi, ²¹³Bi, ²²³Ra, ²²⁴Ra, ²²⁵Ab, ²²⁵Ac, or ²²⁷Th.

A can comprise a chelator that chelates or binds to a radioisotope. The radioisotope can be a therapeutic radioisotope. In certain embodiments, the radioscope is or comprises lutetium (Lu) or indium (In). The radioisotope can be a positron emission tomography (PET)-imaging radioisotope, such as ⁶⁸Ga or a PET-imaging radioisotope selected from the group consisting of ¹¹C, ¹³N, ¹⁵O, and ¹⁸F. The radioisotope can be a therapeutic radioisotope comprising an α-emitting radioisotope or a β-emitting radioisotope. In certain embodiments, the β-emitting radioisotope is ¹⁷⁷Lu.

The chelator can be an imaging radioisotope or a PET-imaging radioisotope. In certain embodiments, the imaging radioisotope is a γ-emitting radioisotope and is ¹¹¹In.

A can comprise a radioisotope selected from the group consisting of ¹⁴C, ³H, ³⁴S, ³²P, ¹²⁵I, and ¹³¹I. The radioactive agent A can be conjugated to the N- or C-terminus of HB or to any active groups on repeating moieties of HB via linker L_{A}.

The conjugate can have one of the following structures:

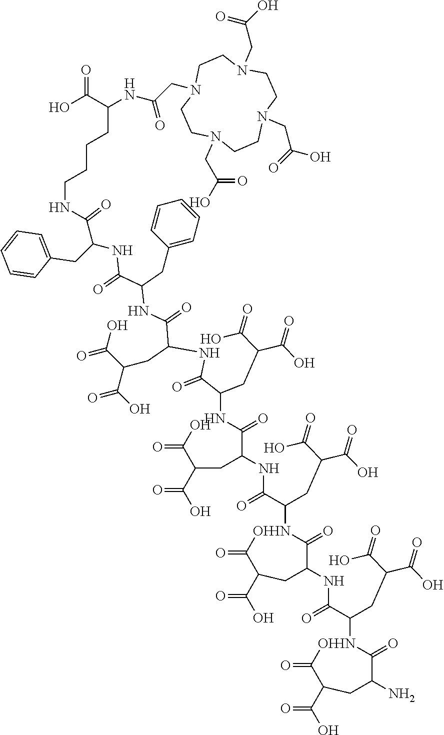 [SMILES: NC(CC(C(=O)O)C(=O)O)C(=O)NC(CC(C(=O)O)C(=O)O)C(=O)NC(CC(C(=O)O)C(=O)O)C(=O)NC(CC(C(=O)O)C(=O)O)C(=O)NC(CC(C(=O)O)C(=O)O)C(=O)NC(CC(C(=O)O)C(=O)O)C(=O)NC(CC(C(=O)O)C(=O)O)C(=O)NC(Cc1ccccc1)C(=O)NC(Cc1ccccc1)C(=O)NCCCCC(NC(=O)CN1CCN(CC(=O)O)CCN(CC(=O)O)CCN(CC(=O)O)CC1)C(=O)O]

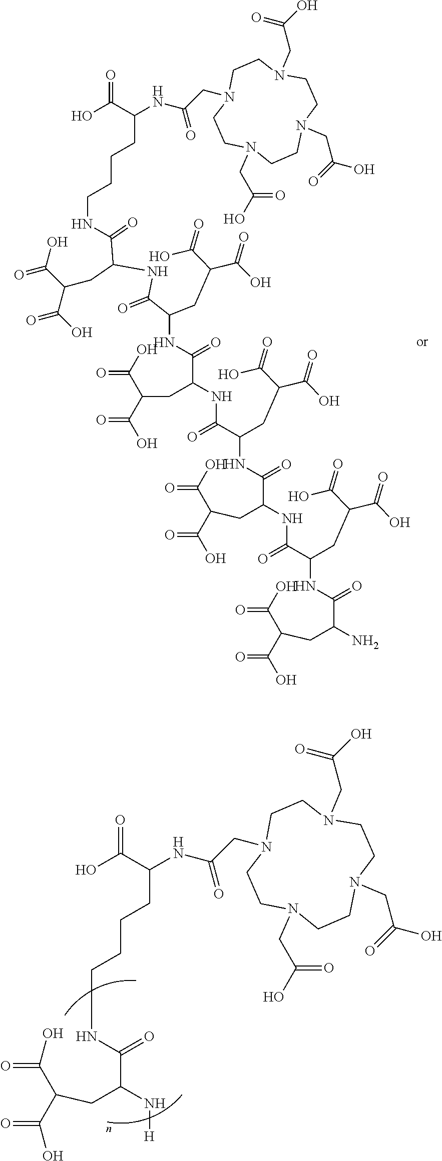

The conjugate can have the following structure:

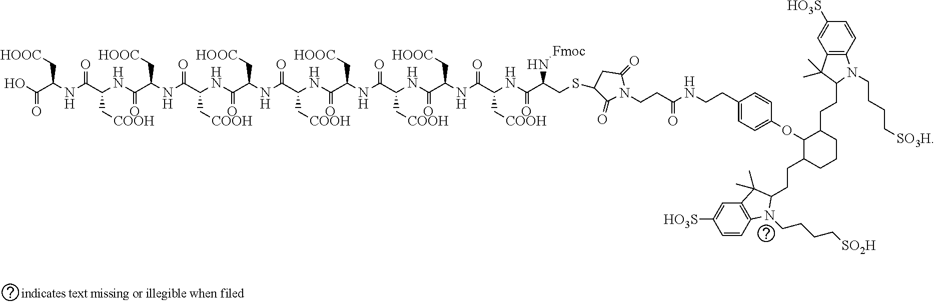

The conjugate can have the following structure:

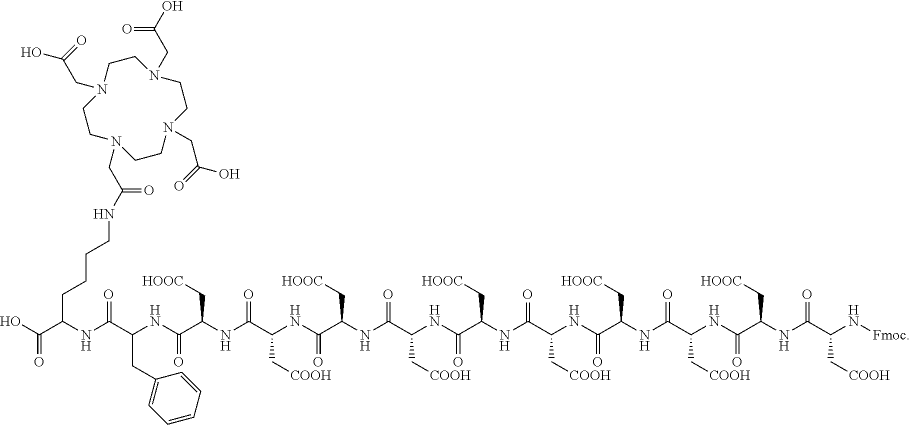

The conjugate can have the following structure:

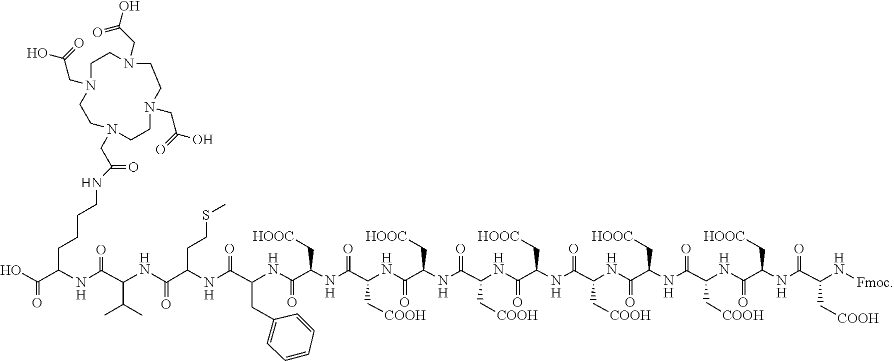

The conjugate can have the following structure:

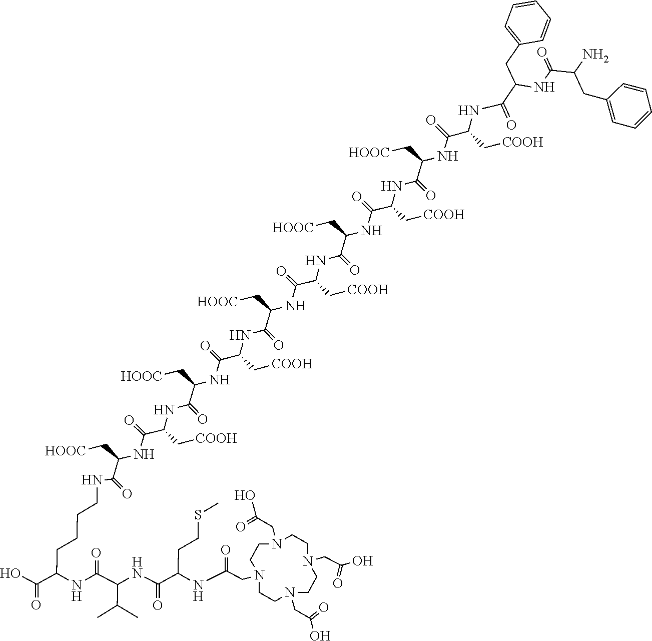 [SMILES: CSCCC(NC(=O)CN1CCN(CC(=O)O)CCN(CC(=O)O)CCN(CC(=O)O)CC1)C(=O)NC(C(=O)NC(CCCCNC(=O)[C@@H](CC(=O)O)NC(=O)[C@@H](CC(=O)O)NC(=O)[C@@H](CC(=O)O)NC(=O)[C@@H](CC(=O)O)NC(=O)[C@@H](CC(=O)O)NC(=O)[C@@H](CC(=O)O)NC(=O)[C@@H](CC(=O)O)NC(=O)[C@@H](CC(=O)O)NC(=O)[C@@H](CC(=O)O)NC(=O)[C@@H](CC(=O)O)NC(=O)C(Cc1ccccc1)NC(=O)C(N)Cc1ccccc1)C(=O)O)C(C)C]

The conjugate can comprise a conjugate chelated to ¹⁷⁷Lu and having the structure:

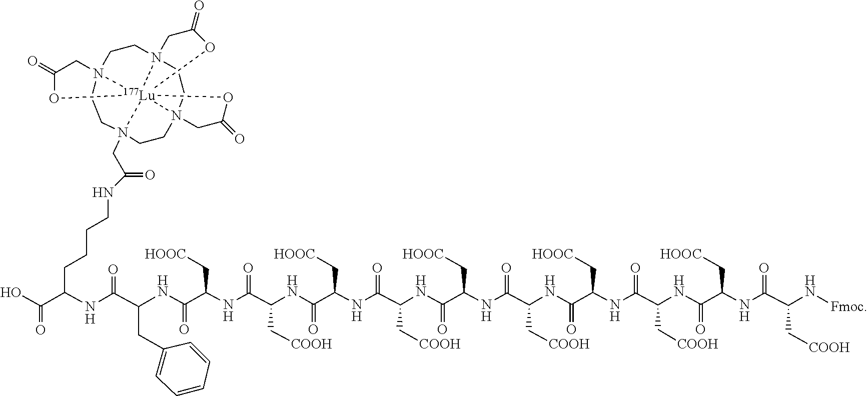

The conjugate can comprise a conjugate chelated to ¹¹¹In and having the structure:

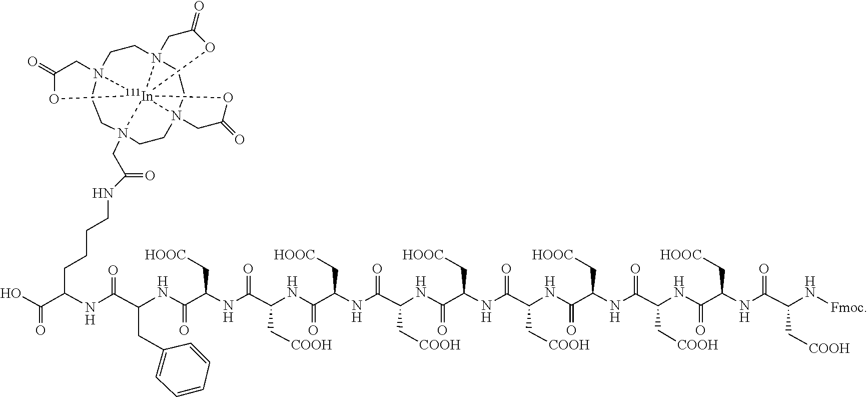

Pharmaceutical compositions are also provided. The pharmaceutical composition comprises a conjugate of Formula I or Formula IA, or a pharmaceutically acceptable sale of Formula I or Formula IA, and a pharmaceutically acceptable carrier or excipient. In certain embodiments, the pharmaceutical composition comprises:
a conjugate of Formula I:

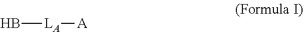

or is a pharmaceutically acceptable salt thereof, wherein:
HB comprises a radical of a D-γ-carboxyglutamic acid ligand, L-γ-carboxyglutamic acid ligand, or a mixture of the foregoing or derivatives thereof that binds hydroxyapatite,
A is an active agent comprising a radio-imaging agent, a radio-sensitizing agent, a radio-protecting agent, or a radiotherapeutic agent, and
L_{A} is a linker that binds HB and A, or absent; and
   a pharmaceutically acceptable carrier or excipient.

The pharmaceutical composition can further comprise a radiosensitizer, a radioprotector, an immunotherapeutic agent, a chemotherapeutic agent, an anti-cancer agent, and/or a hormone therapeutic agent.

Further provided is a method of imaging and/or treating a bone in a subject. In certain embodiments, the method comprises administering to the subject an effective amount (e.g., a therapeutically effective amount) of: (i) a conjugate or pharmaceutically acceptable salt thereof described herein, a first pharmaceutical composition described herein (e.g., comprising the conjugate or pharmaceutically acceptable salt thereof), or (ii) a first pharmaceutical composition comprising a conjugate of Formula I or IA and a first pharmaceutically acceptable carrier or excipient and simultaneously or sequentially, in either order, (b)(i′) an active agent (e.g., a free drug), or (ii′) a second pharmaceutical composition comprising the active agent and a second pharmaceutically acceptable carrier or excipient. The active agent of (b) can be/comprise a radiosensitizer, radioprotector, immunotherapeutic agent, chemotherapeutic agent, anti-cancer drug, or hormone therapeutic agent. The active agent of (b) can comprise lysine (Lys). In certain embodiments, HB comprises a radical of a D-γ-carboxyglutamic acid ligand, L-γ-carboxyglutamic acid ligand, or a mixture of the foregoing or derivatives thereof. In certain embodiments, HB comprises 7-20 D-γ-carboxyglutamic acid residues.

The subject can have cancer, such as primary bone cancer, e.g., osteosarcoma, chondrosarcoma, Ewing sarcoma, or chordoma, or secondary bone cancer, e.g., metastatic breast cancer, prostate cancer, multiple myeloma, thyroid cancer, lung cancer, kidney cancer, ovarian cancer, colon cancer, or melanoma.

The method can further comprise imaging the bone of the subject. When the bone in the subject is imaged, the method can further comprise diagnosing whether the subject has cancer. When the subject has been treated for cancer and the bone in the subject is imaged, the method can further comprise assessing or monitoring the efficacy of treatment.

A method of binding a conjugate or a pharmaceutically acceptable salt thereof to hydroxyapatite in a subject is also provided. Such method can comprise administering to the subject an effective amount of: (i) a conjugate or a pharmaceutically acceptable salt thereof; or (ii) a pharmaceutical composition (e.g., comprising the conjugate or a pharmaceutically acceptable salt thereof). In some embodiments, the subject has cancer (e.g., bone cancer). In some embodiments, the subject has an osteoblastic bone cancer. In some embodiments, the subject has an osteolytic bone cancer.

### DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 shows luciferin bioluminescence in the right femur of a mouse two weeks after initial tumor challenge, evidencing tumor growth in the bone (bioluminescence circled).

FIG. 2 shows a micro computed tomography (microCT) image of a mouse two weeks after initial tumor challenge in the right tibia, evidencing severe bone resorption due to tumor growth.

FIG. 3 shows fluorescence of DD10-S0456 localized to tumor-induced bone lesions in the mouse shown in FIG. 1 (fluorescence circled).

FIG. 4 shows the reaction scheme of the fluorescence-imaging conjugate DD10-S0456, with the following additions/protocols at each identified step as follows: (1) 1 eq 50456 and 5 eq CsCO₃ in dimethyl sulfoxide (DMSO) at room temperature (RT) for 4 hours; (2) a) Boc deprotection 40% trifluoroacetic acid (TFA) in dichloromethane (DCM) at RT for 40 minutes; and b) 1 eq 3-maleimidopropionic acid, 1 eq benzotriazole-1-yloxytripyrrolidinophosphonium hexafluorophosphate (PyBOP), and 5 eq N,N-Diisopropylethylamine (DIPEA) in anhydrous DMSO at RT for 4 hours; and (3) 1 eq DD10-Cys-Fmoc in anhydrous DMSO at RT for 4 hours.

FIG. 5 shows a reaction scheme of the radio-imaging and radiotherapeutic conjugates K(DOTA)-V-M-F-DD10 and K(DOTA)-F-DD10. While DOTA are the chelators shown in this scheme, it will be appreciated that other chelators with like functional groups can be attached in a similar manner.

FIG. 6 shows the structure of Lys(DOTA)-Phe-D-Asp₁₀ (also referred to as K(DOTA)-F-DD10 or KDFDD10).

FIG. 7 shows the structure of Lys(DOTA)-Val-Met-Phe-D-Asp₁₀ (also referred to as K(DOTA)-V-M-F-DD10 or KDVMFDD10).

FIG. 8 shows the single-photon emission computer tomography/computed tomography (SEPCT/CT) images of a tibial tumor bearing mouse injected with ¹¹¹In chelated K(DOTA)-F-DD10 (220 μCi of 200 μCi/10 nmol dosage injected). The arrows indicate the site of tumor growth, and the white boxes indicate the kidney. From left to right: imaged at 1 hour, 4 hours, 12 hours, 24 hours, 48 hours, 72 hours, 96 hours, and 120 hours post radio-imaging agent injection.

FIG. 9 shows the single photon emission computer tomography (SPECT)/computed tomography (CT) images of a tibial tumor bearing mouse injected with ¹¹¹In chelated K(DOTA)-V-M-F-DD10 (375 μCi of 300 μCi/10 nmol dosage injected). The arrows indicate the site of tumor growth, and the white boxes indicate the kidney. From left to right: imaged at 1 hour, 4 hours, 12 hours, 24 hours, 48 hours, 72 hours, 96 hours, and 120 hours post radio-imaging agent injection.

FIG. 10 shows the SPECT/CT images of healthy mice injected with ¹¹¹In chelated K(DOTA)-F-DD10 (160 μCi of 200 μCi/10 nmol dosage injected) in the top row and with ¹¹¹In chelated K(DOTA)-V-M-F-DD10 (350 μCi of 300 μCi/10 nmol dosage injected) in the bottom row. Arrows indicate the kidney. From left to right: imaged at 1 hour, 4 hours, 12 hours, 24 hours, 48 hours, 72 hours, 96 hours, and 120 hours post radio-imaging agent injections.

FIG. 11 shows the SPECT/CT images of a tibial tumor bearing mouse injected with ¹⁷⁷Lu chelated K(DOTA)-F-DD10 (0.962 mCi of 1.5 mCi/10 nmol dosage injected). The arrows indicate the site of tumor growth, and the white boxes indicate the kidney. From left to right: imaged at 3 hours, 24 hours, 48 hours, 72 hours, 96 hours, 120 hours, and 168 hours post radiotherapeutic agent injection.

FIG. 12 shows the SPECT/CT images of a tibial tumor bearing mouse injected with ¹⁷⁷Lu chelated K(DOTA)-F-DD10 (3.0 mCi of 3.0 mCi/10 nmol dosage injected). The arrows indicate the site of tumor growth, and the white boxes indicate the kidney. From left to right: imaged at 3 hours, 24 hours, 48 hours, 72 hours, 96 hours, 120 hours, and 168 hours post radiotherapeutic agent injection.

FIG. 13 shows the SPECT/CT images of a tibial tumor bearing mouse injected with ¹⁷⁷Lu chelated K(DOTA)-F-DD10 (3.57 mCi of 3.0 mCi/10 nmol dosage injected). The arrows indicate the site of tumor growth, and the white boxes indicate the kidney. Top row from left to right: imaged at 2 hours, 12 hours, 24 hours, and 48 hours post radiotherapeutic agent injection. Bottom row from left to right: imaged at 72 hours, 96 hours, 144 hours, and 196 hours post radiotherapeutic injection.

FIG. 14 shows the SPECT/CT images of a tibial tumor bearing mouse injected with ¹⁷⁷Lu chelated K(DOTA)-F-DD10 (5.50 mCi of 6.0 mCi/10 nmol) dosage injected). The arrows indicate the site of tumor growth, and the white boxes indicate the kidney. Top row from left to right: imaged at 2 hours, 12 hours, 24 hours, 48 hours, and 72 hours post radiotherapeutic agent injection. Bottom row from left to right: imaged at 96 hours, 144 hours, 196 hours, and 240 hours post radiotherapeutic agent injection.

FIG. 15 shows a reaction scheme of the radio-imaging and radiotherapeutic conjugate K(DD10F2)VM-DOTA, building from carboxyl end to amine end. Steps 1-4 identified under the third arrow comprise: 1) Alloc deprotection; 2) KVM-DOTA+DD10F2-Fmoc coupling; 3) Fmoc deprotection; and 4) Deprotection and cleavage.

FIG. 16 shows a structure of Lys(D-Asp₁₀-Phe₂)-Val-Met-DOTA (also referred to as K(DD10F2)VM-DOTA).

FIG. 17 shows a structure of a ¹⁷⁷Lu-complexed DOTA conjugate, wherein the underlying conjugate comprises the conjugate of FIG. 6.

FIG. 18 shows a structure of a ¹¹¹In-complexed DOTA conjugate, wherein the underlying conjugate comprises the conjugate of FIG. 6.

FIG. 19 shows a structure of a D-Ga-(D-Gla)ₙ-Cys-S0456 conjugate, wherein n=1-50.

FIG. 20 shows a structure of a D-Gla-(D-Gla)ₙ-Phe-DOTA conjugate, wherein n=1-50.

FIG. 21 shows a structure of a S0456-Cys-GYK-Phe-(D-Gla)ₙ conjugate, wherein n=1-50.

FIG. 22 shows a structure of a DOTA-GYK-Phe-(D-Gla)ₙ conjugate, wherein n=1-50.

FIG. 23 shows a structure of a S0456-Cys-GYK-Phe-(D-Gla)₁₄ conjugate.

FIG. 24 shows a structure of a DOTA-GYK-Phe-(D-Gla)₁₄ conjugate.

FIG. 25 shows a synthesis scheme of a bone-targeted D-Gla peptide conjugated to a DOTA.

FIG. 26 shows a graph of comparative biodistribution of the injected conjugates 48 hours post-injection in various organs. Uptake is expressed as percent injected dose per gram (% ID/g), with tumor uptake calculated using the contralateral tibia excluding the joint regions.

FIG. 27 shows a graph of comparative biodistribution of the injected conjugates 24 hours post-injection in various organs. Uptake is expressed as % ID/g, with tumor % ID/g values calculated using weights of marrow extracted from the tumor-bearing tibias, and healthy tibia % ID/g values calculated using the contralateral tibia excluding the joint regions.

While the present disclosure is susceptible to various modifications and alternative forms, exemplary embodiments thereof are shown by way of example in the drawings and are herein described in detail.

### DETAILED DESCRIPTION

The present disclosure is predicated, at least in part, on the use of polyanionic, poly-acidic, and/or polyelectrolytic polymers, such as polymers comprising or consisting of acidic amino acids (e.g., L and/or D, aspartic and/or glutamic acids) to target hydroxyapatite (e.g., the positively charged calcium heads of hydroxyapatite). Provided are conjugates comprising a radical of a polyanionic, polyacidic, and/or polyelectrolytic ligand (e.g., the carboxyl ends, amino ends, and/or carboxylic side chains of acidic peptides), which binds hydroxyapatite, coupled with an active agent, such as an imaging, radiotherapeutic, radio-sensitizing, and/or radio-protecting agent. In certain embodiments, the attachment of a polyanionic, poly-acidic and/or poly-electrolytic ligand to the active agent can be via a linker (optionally further including a spacer). Such conjugates can be used to target delivery of the aforementioned active agents to damaged or diseased bone, such as bone comprising a cancer-induced lesion, for diagnostic and/or therapeutic (e.g., “theragnostic”) purposes.

When cancer cells invade bone, they excrete various compounds (such as cytokines or growth factors) to induce osteoclasts (i.e., bone-degrading cells) to hyper-proliferate and activate. The osteoclasts can then work to deteriorate the bone and assist in tumor growth by releasing various growth factors and compounds that were previously locked in the bone matrix. During this process of bone resorption, hydroxyapatite can be exposed to blood vessels and become targetable. While hydroxyapatite may be exposed in normal healthy bones as bones are continuously undergoing turnover to maintain bone homeostasis (a balance between formation and degradation), the exposure is insignificant as compared to the exposure in diseased or damaged bones. Additionally, unlike other receptors, exposed hydroxyapatite is highly abundant and thus does not become readily saturated by clinically therapeutic dosages. These characteristics render hydroxyapatite a potential receptor for selective delivery of active agents.

### Conjugates

The conjugates can bind to hydroxyapatite. The conjugates can deliver imaging, radiotherapeutic, radio-sensitizing, and/or radio-protecting agents to damaged or diseased bone, such as bone comprising cancer-induced lesions, for theragnostic purposes.

In certain embodiments, a conjugate comprises Formula I:

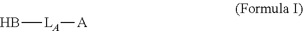

or a pharmaceutically acceptable salt thereof, wherein HB is a radical a D-γ-carboxyglutamic acid ligand, L-γ-carboxyglutamic acid ligand, or a mixture of the foregoing or derivatives thereof (e.g., that binds hydroxyapatite); A is an active agent comprising a radio-imaging agent, a radio-sensitizing agent, a radio-protecting agent, or a radiotherapeutic agent; and, optionally, L_{A} is a linker that binds HB and A, or is absent.

In certain embodiments, a conjugate comprises Formula I:

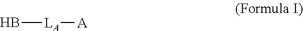

or a pharmaceutically acceptable salt thereof, wherein HB is a radical of an anionic, acidic, and/or electrolytic ligand (e.g., that binds hydroxyapatite); A is an active agent comprising a radio-imaging agent, a radio-sensitizing agent, a radio-protecting agent, or a radiotherapeutic agent; and, optionally, L_{A} is a linker that binds HB and A, or is absent.

In certain embodiments, L_{A} is optional and, where not included, HB is conjugated directly to A. For example, in certain embodiments, a conjugate comprises Formula IA:

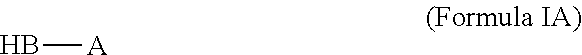

or a pharmaceutically acceptable salt thereof, wherein HB is a radical of an anionic, acidic, and/or electrolytic ligand (e.g., that binds hydroxyapatite); and A is an active agent comprising a radio-imaging agent, a radio-sensitizing agent, a radio-protecting agent, or a radiotherapeutic agent.

In certain embodiments, the D-γ-carboxyglutamic acid ligand, L-γ-carboxyglutamic acid ligand, or a mixture of the foregoing or derivatives thereof (e.g., HB) is a straight-chain (e.g., non-branched) ligand. In certain embodiments, the D-γ-carboxyglutamic acid ligand, L-γ-carboxyglutamic acid ligand, or a mixture of the foregoing or derivatives thereof is a branched ligand.

In certain embodiments, the anionic, acidic, and/or electrolytic ligand is a straight-chain (e.g., non-branched) anionic, acidic, and/or electrolytic ligand. In certain embodiments, the anionic, acidic, and/or electrolytic ligand is a branched-chain anionic, acidic, and/or electrolytic ligand.

The ligand (e.g., HB) can be a biological targeting moiety which has one or more negative charges or can otherwise interact with positively charged calcium of hydroxyapatites. In certain embodiments, the ligand comprises peptides or peptide analogs (which can be linear peptides, cyclic peptides, branched peptides, or combinations thereof) and/or one or more polymers. The ligand can be of synthetic or natural origin. In certain embodiments, HB comprises an amino acid or derivative thereof, and at least one third of the amino acid sequence thereof is acidic in nature.

The ligand (e.g., HB) can comprise from one to about 50 (e.g., 1-50) moieties (e.g., anionic, acidic, and/or electrolytic moieties). In certain embodiments, HB is or comprises one to about 100 (e.g., 1-100) D-γ-carboxyglutamic acid residues, L-γ-carboxyglutamic acid residues, or a mixture of the foregoing or derivatives thereof. In certain embodiments, HB is or comprises at or about 5 to at or about 95 D-γ-carboxyglutamic acid residues, L-γ-carboxyglutamic acid residues, or a mixture of the foregoing or derivatives thereof. In certain embodiments, HB is or comprises at or about 10 to at or about 90 D-γ-carboxyglutamic acid residues, L-γ-carboxyglutamic acid residues, or a mixture of the foregoing or derivatives thereof. In certain embodiments, HB is or comprises at or about 15 to at or about 85 D-γ-carboxyglutamic acid residues, L-γ-carboxyglutamic acid residues, or a mixture of the foregoing or derivatives thereof. In certain embodiments, HB is or comprises at or about 20 to at or about 80 D-γ-carboxyglutamic acid residues, L-γ-carboxyglutamic acid residues, or a mixture of the foregoing or derivatives thereof. In certain embodiments, HB is or comprises at or about 25 to at or about 75 D-γ-carboxyglutamic acid residues, L-γ-carboxyglutamic acid residues, or a mixture of the foregoing or derivatives thereof. In certain embodiments, HB is or comprises at or about 30 to at or about 70 D-γ-carboxyglutamic acid residues, L-γ-carboxyglutamic acid residues, or a mixture of the foregoing or derivatives thereof. In certain embodiments, HB is or comprises at or about 35 to at or about 65 D-γ-carboxyglutamic acid residues, L-γ-carboxyglutamic acid residues, or a mixture of the foregoing or derivatives thereof. In certain embodiments, HB is or comprises at or about 40 to at or about 60 D-γ-carboxyglutamic acid residues, L-γ-carboxyglutamic acid residues, or a mixture of the foregoing or derivatives thereof. In certain embodiments, HB is or comprises at or about 45 to at or about 55 D-γ-carboxyglutamic acid residues, L-γ-carboxyglutamic acid residues, or a mixture of the foregoing or derivatives thereof. In certain embodiments, HB is or comprises at or about 50 D-γ-carboxyglutamic acid residues, L-γ-carboxyglutamic acid residues, or a mixture of the foregoing or derivatives thereof.

In certain embodiments, HB is or comprises 7-20 D-γ-carboxyglutamic acid residues. In certain embodiments, HB is or comprises 7-20 L-γ-carboxyglutamic acid residues. In certain embodiments, HB is or comprises 7 D-γ-carboxyglutamic acid residues. In certain embodiments, HB is or comprises 7 L-γ-carboxyglutamic acid residues.

In certain embodiments, the ligand comprises a polyacid ligand. A polyacid ligand comprises repeating units bearing acidic moieties. Examples of acidic moieties include, but are not limited to, carboxylic, sulfonic, boronic, and phosphonic moieties. An acidic moiety can be an acidic macromolecule, such as a nucleic acid, an actin, or a proteoglycan. In certain embodiments, the ligand (e.g., HB) comprises a polyacidic peptide and/or polymer such as, for example, a polyglutamic acid peptide and the like. In certain embodiments, the ligand comprises a polyelectrolytic ligand. A polyelectrolytic ligand can comprise repeating units bearing electrolytic moieties. Examples of electrolytic moieties include, but are not limited to, sulfonates, acrylates, and phosphates.

In certain embodiments, the ligand comprises from one to about 50 (e.g., 1-50) anionic moieties. In certain embodiments, the ligand comprises a polyanion. A polyanion is a subgroup of polyelectrolytes comprised of repeating anionic moieties (e.g., moieties based on a polymer). Examples of anionic moieties include, but are not limited to, phosphate and polyphosphate.

The ligand (e.g., HB) can comprise an amino acid or a derivative thereof. As used herein, “amino acid” means an L- or D-amino acid, amino acid analog, or amino acid mimetic that can be naturally occurring or of purely synthetic origin, and can be optionally pure (i.e., a single enantiomer and hence chiral, or a mixture of enantiomers). As used herein, “amino acid mimetic” means synthetic analogs of naturally occurring amino acids which are isosteres (i.e., have been designed to mimic the steric and electronic structure of the natural compound). The amino acid can be natural or unnatural, essential or non-essential. The amino acid can have any suitable relative configuration (such as a D- or L-configuration, described above). The HB can comprise L-aspartic acid, D-aspartic acid, L-glutamic acid, D-glutamic acid, D-γ-carboxyglutamic acid, L-γ-carboxyglutamic acid, or a mixture of two or more of the foregoing or derivatives thereof. In certain embodiments, the HB comprises the structure:

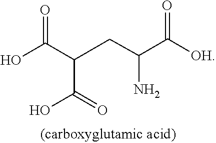 [SMILES: NC(CCC(C(=O)O)C(=O)O)C(=O)O]

The ligand (e.g., HB) can further comprise one or more hydrophobic moieties coupled with the radical of an anionic, acidic, and/or electrolytic ligand, such as from one to about 30 (e.g., 1-30) hydrophobic moieties, such as an alkyl or an alkenyl, ultraviolet (UV)-active moieties, such as phenylalanine (Phe), tyrosine (Tyr) and/or tryptophan (Trp), or active group-protecting moieties, such as 9-fluorenylmethoxycarbonyl (Fmoc), an allyloxycarbonyl (Alloc), and/or t-butyl. “Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈ alkyl). An alkyl can comprise two to five carbon atoms (e.g., C₂-C₅ alkyl). An alkyl can comprise three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond.

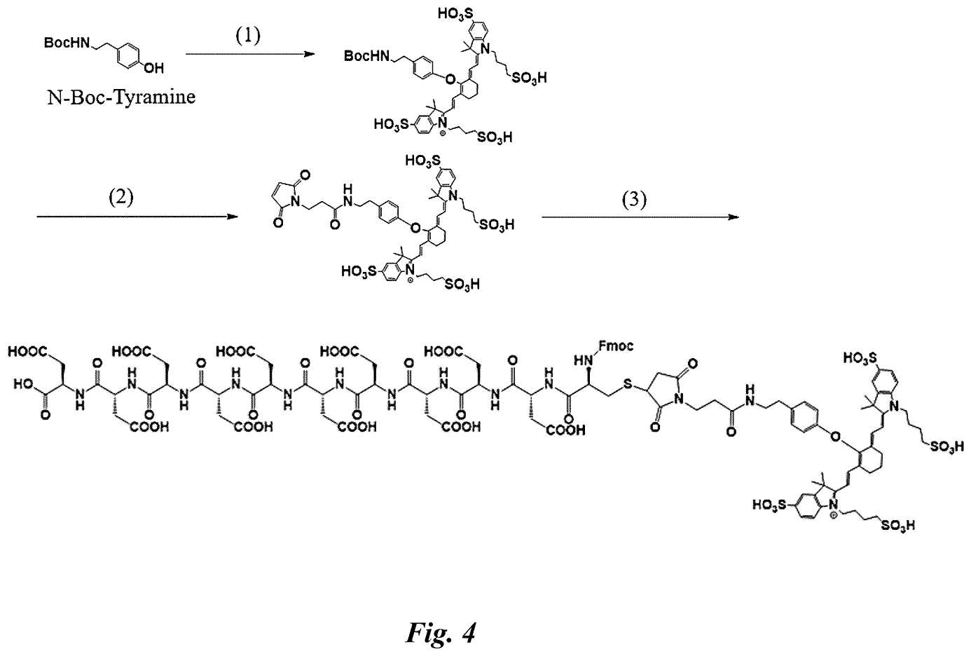

The ligand (e.g., HB) can further comprise (i) a serum albumin binder (AB) conjugated directly to HB, or (ii) AB and a linker L_{AB} (AB-L_{AB}), wherein AB-L_{AB} is conjugated to HB via L_{AB}. In certain embodiments, AB is attached to an amine or a carboxyl end of a polyacidic peptide or an active or functional group (such as, by way of non-limiting example, a carboxyl, an amine, or a thiol group) of the repeating anionic, acidic, or electrolytic moieties of HB. AB can be attached directly to HB or can be attached via a linker L_{AB}. In certain embodiments, AB is attached to a thiol of HB by using a maleimide linker (e.g., see the maleimide-thiol linker of FIG. 4 used, in that instance, to conjugate a dye to the targeting moiety), a disulfide linker, or any other linker that can react with a sulfur group. In some embodiments, HB does not contain AB.

Examples of AB include, but are not limited to, Evans Blue, Fmoc, diphenylcyclohexanol phosphate ester, naphthalene acyl sulfonamide, 4-(p-X-phenyl) butyric acid, in which X is —H, —C₁-C₆ alkyl, —F, —Cl, —Br, —I, —O—C₁₋₆alkyl, —CN, —CHO, —B(OH)₂, —C═C—C(O)aryl, —C═C—S(O)₂aryl, —CO₂H, —SO₃H, —SO₂NH₂, —PO₃H₂, —SO₂F, CF₃, or a derivative of any of the foregoing. Additional examples of AB include, but are not limited to:

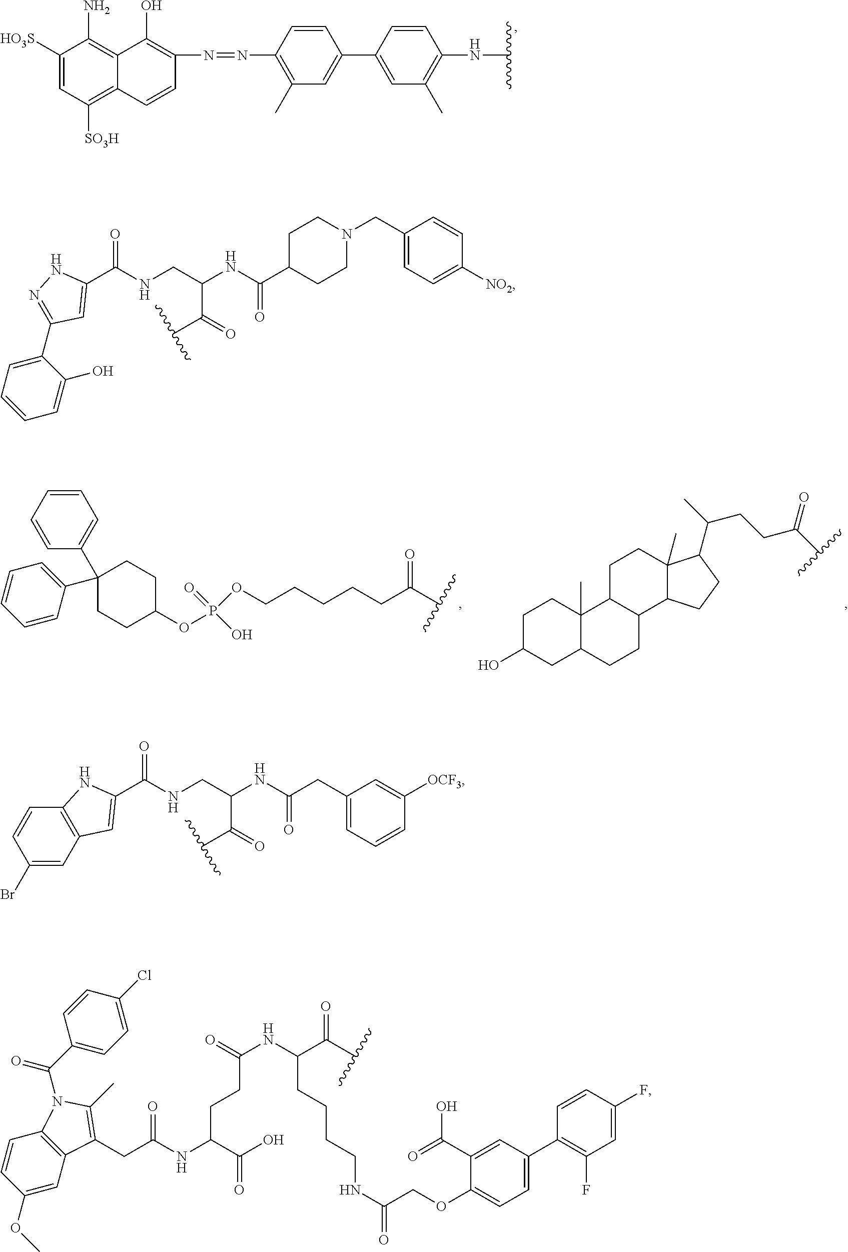 [SMILES: *Nc1ccc(-c2ccc(N=Nc3ccc4c(S(=O)(=O)O)cc(S(=O)(=O)O)c(N)c4c3O)c(C)c2)cc1C ; *C(=O)C(CNC(=O)c1cc(-c2ccccc2O)n[nH]1)NC(=O)C1CCN(Cc2ccc([N+](=O)[O-])cc2)CC1 ; *C(=O)CCCCCOP(=O)(O)OC1CCC(c2ccccc2)(c2ccccc2)CC1 ; *C(=O)CCC(C)C1CCC2C3CCC4CC(O)CCC4(C)C3CCC12C ; *C(=O)C(CNC(=O)c1cc2cc(Br)ccc2[nH]1)NC(=O)Cc1cccc(OC(F)(F)F)c1 ; *C(=O)C(CCCCNC(=O)COc1ccc(-c2ccc(F)cc2F)cc1C(=O)O)NC(=O)CCC(NC(=O)Cc1c(C)n(C(=O)c2ccc(Cl)cc2)c2ccc(OC)cc12)C(=O)O]

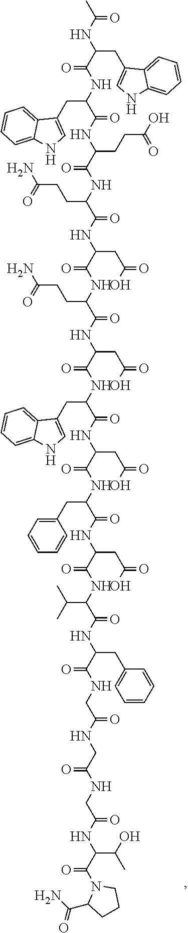 [SMILES: CC(=O)NC(Cc1c[nH]c2ccccc12)C(=O)NC(Cc1c[nH]c2ccccc12)C(=O)NC(CCC(=O)O)C(=O)NC(CCC(N)=O)C(=O)NC1CC(=O)ON(C(CCC(N)=O)C(=O)NC2CC(=O)ON(C(Cc3c[nH]c4ccccc34)C(=O)NC3CC(=O)ON(C(Cc4ccccc4)C(=O)NC4CC(=O)ON(C(C(=O)NC(Cc5ccccc5)C(=O)NCC(=O)NCC(=O)NCC(=O)NC(C(=O)N5CCCC5C(N)=O)C(C)O)C(C)C)C4=O)C3=O)C2=O)C1=O]

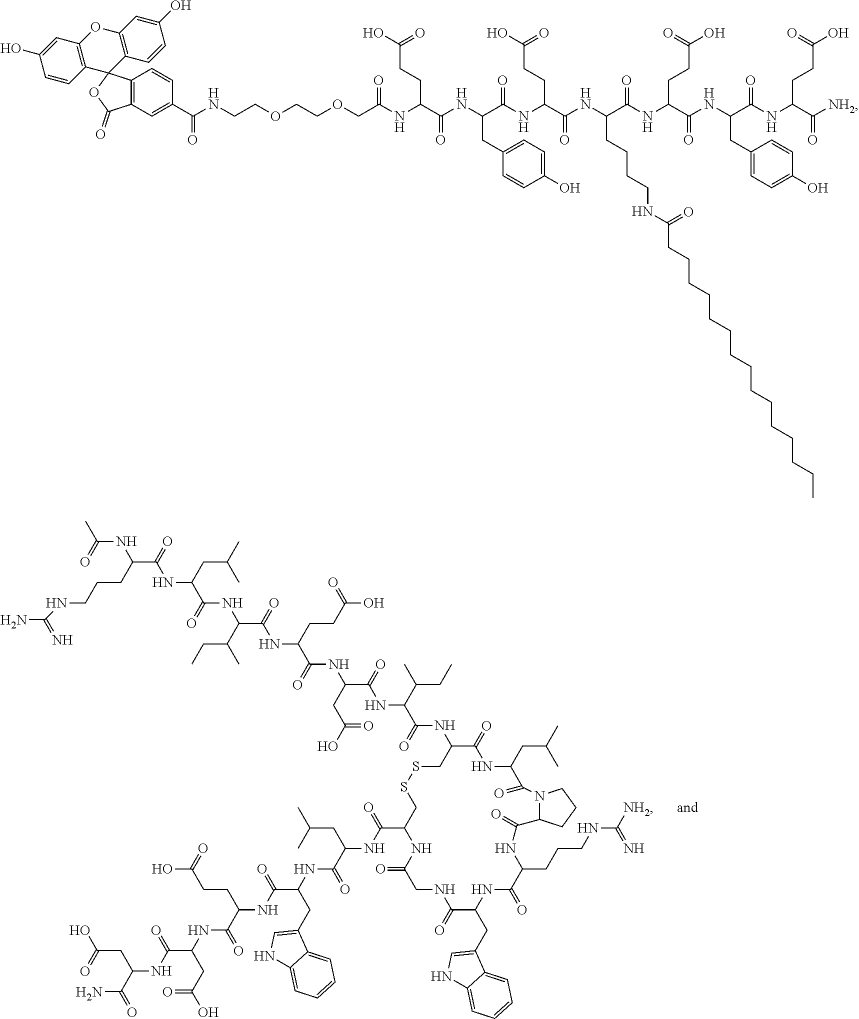

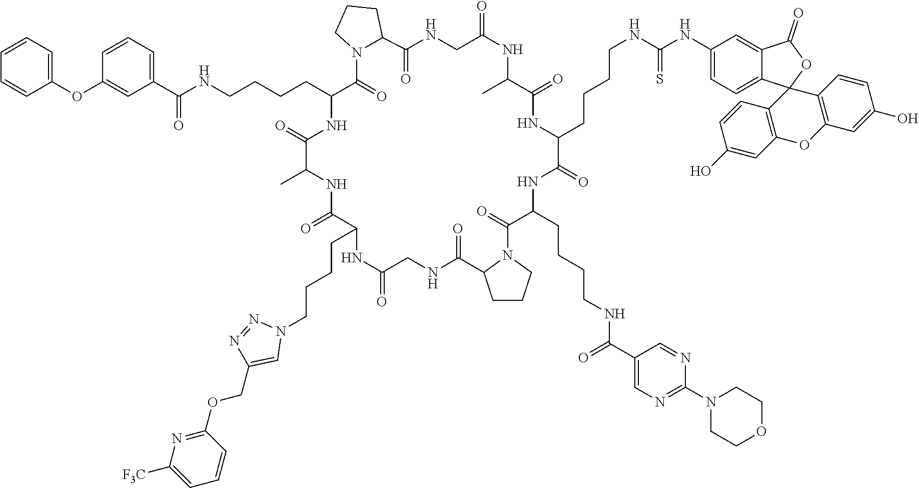 [SMILES: CC1NC(=O)CNC(=O)C2CCCN2C(=O)C(CCCCNC(=O)c2cccc(Oc3ccccc3)c2)NC(=O)C(C)NC(=O)C(CCCCn2cc(COc3cccc(C(F)(F)F)n3)nn2)NC(=O)CNC(=O)C2CCCN2C(=O)C(CCCCNC(=O)c2cnc(N3CCOCC3)nc2)NC(=O)C(CCCCNC(=S)Nc2ccc3c(c2)C(=O)OC32c3ccc(O)cc3Oc3cc(O)ccc32)NC1=O]

AB can comprise a hapten. Examples of haptens include, but are not limited to, 2,4-dinitrophenol (DNP), 2,4,6-trinitrophenol (TNP), rhamnose, galactose-α-1,3-galactose (α-Gal), or an antibody binder. Examples of antibody binders include, but are not limited to, a Fab, an scFv, a V_{H}, a V_{L}, a V_{H}H, a V-NAR, a monobody, an anticalin, an affibody, and a DARPin.

HB can comprise a radical of a straight-chain polyanion of formula X-A₁, X-A₂ X-A₃, or X-A₄:

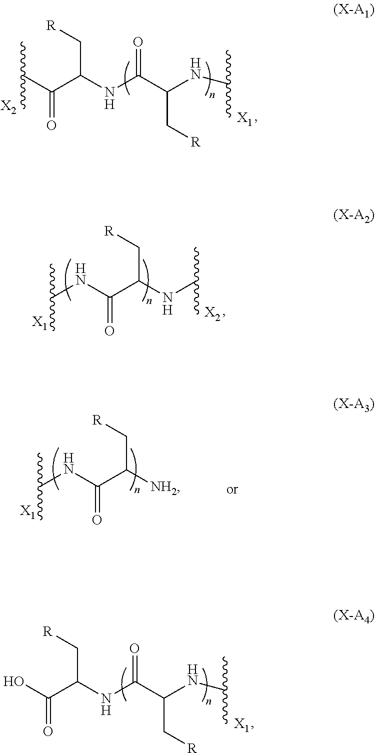

wherein:
R is COOH for L- or D-aspartic acid, CH₂COOH for L- or D-glutamic acid, HOOC—CH₂—CH(COOH)—CH(NH₂)—COOH or D- or L-γ-carboxyglutamic acid, or CH₂CH₂COOH for an unnatural, acidic amino acid;
n=1-50; and

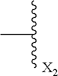

represents the point of attachment to AB or L_{AB}.

In some embodiments, HB does not comprise an albumin binder, as shown in formulae X-A₃ and X-A₄. HB can comprise a radical of a branched-chain polyanion of formula X-A₅, X-A₆, X-A₇, or X-A₈:

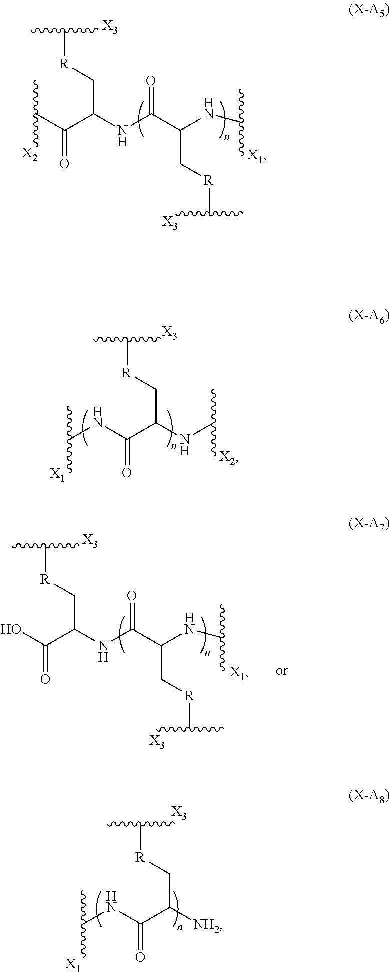

wherein:
R is COO— for L- or D-aspartic acid, CH₂COO— for L- or D-glutamic acid, HOOC—CH₂—CH(COOH)—CH(NH₂)—COOH or D- or L-γ-carboxyglutamic acid, or CH₂CH₂COO— for an unnatural, acidic amino acid;
n=1-50;

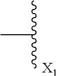

represents the point of attachment to L_{A}; and,

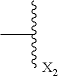

when HB further comprises AB or AB-L_{AB},
   represents the point of attachment to AB or L_{AB}, and

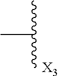

represents the point of attachment to hydrogen or an HB. In certain embodiments, HB does not comprise any albumin binders as shown in formulae X-A₇ and X—As.

Linkers, represented by L_{A} and L_{AB}, can be any suitable linkers. The linker can comprise atoms selected from C, N, O, S, Si, and P; C, N, O, S, and P; or C, N, O, and S. The linker can have a backbone that ranges in length, such that there can be as few as two atoms in the backbone of the linker to as many as 100 or more contiguous atoms in the backbone of the linker. The “backbone” of the linker is the shortest chain of contiguous atoms forming a covalently bonded connection between HB and A. In some embodiments, a polyvalent linker has a branched backbone, with each branch serving as a section of backbone linker until reaching a terminus.

For example, the linker can have a chain length of at least about 7 atoms. In some embodiments, the linker is at least about 10 atoms in length. In some embodiments, the linker is at least about 14 atoms in length. In some embodiments, the linker is between about 7 and about 31 atoms (such as, about 7 to 31, 7 to about 31, or 7 to 31), between about 7 and about 24 atoms (such as, about 7 to 24, 7 to about 24, or 7 to 24), or between about 7 and about 20 atoms (such as, about 7 to 20, 7 to about 20, or 7 to 20) atoms. In some embodiments, the linker is between about 14 and about 31 atoms (such as, about 14 to 31, 14 to about 31, or 14 to 31), between about 14 and about 24 atoms (such as, about 14 to 24, 14 to about 24, or 14 to 24), or between about 14 and about 20 atoms (such as, about 14 to 20, 14 to about 20, or 14 to 20). In some embodiments, the linker has a chain length of at least 7 atoms, at least 14 atoms, at least 20 atoms, at least 25 atoms, at least 30 atoms, or at least 40 atoms; or from 1 to 15 atoms, 1 to 5 atoms, 5 to 10 atoms, 5 to 20 atoms, 10 to 40 atoms, or 25 to 100 atoms.

L_{A} and L_{AB} can comprise at least one carbon-carbon bond and/or at least one amide bond. L_{A} and L_{AB} can comprise one or more L- or D-configurations, natural or unnatural amino acids, a polyethylene glycol (PEG) monomer, a PEG oligomer, a PEG polymer, or a combination of any of the foregoing. For a linker that comprises one or more PEG units, all carbon and oxygen atoms of the PEG units are part of the backbone, unless otherwise specified.

L_{A} and L_{AB} can comprise an oligomer of peptidoglycans, glycans, anions, or a combination of any of the foregoing. L_{A} and L_{AB} can comprise at least one 2,3-diaminopropionic acid group, at least one glutamic acid group, at least one cysteine group, or a combination of two or more of the foregoing.

In certain embodiments, L_{A} and/or L_{AB} comprise one or more amino acids. In certain embodiments, L_{A} and/or L_{AB} comprise an amino acid linker (e.g., a lysine (Lys) linker as described in Example 4 below).

L_{A} and/or L_{AB} can each independently be a quick-release linker. L_{A} and/or L_{AB} can each independently be a slow-release linker. As used herein, the term “quick-release” in the context of a linker means a linker that includes at least one bond that is releasable as is known in the art and/or that can be cleaved to varying degrees under certain conditions and, in particular, can be fragmented or cleaved in less than about 1 week when under, or otherwise exposed to, certain metabolic, physiological, or cellular conditions that may initiate a cascade of fragmentation or bond cleavage (which may, for example, result in the release of one or more of the moieties connected through one or more portions of the linker (e.g., HB and A)). Bond cleavage can occur by standard chemical hydrolysis reactions that occur, for example, at physiological pH, or as a result of compartmentalization into a cellular organelle such as an endosome having a lower pH than cytosolic pH. Bond cleavage can also occur by acid catalyzed elimination. Alternatively, fragmentation can be initiated by a nucleophilic attack on a disulfide group of the quick-release linker, causing cleavage to form a thiolate, for example. In any of these cases, the quick-release nature of such linkers can be realized by whatever mechanism may be relevant to the chemical, metabolic, physiological, or biological conditions present. In certain embodiments, a quick-release linker comprises one or more sulfide bridges.

In contrast, a “slow-release” in the context of a linker means a linker that includes at least one bond that is not easily or quickly broken (i.e. the bond does not cleave) and, while potentially cleavable or fragmentable to varying degrees under certain conditions, does not cleave, fragment, or otherwise release one or more of the moieties connected through one or more portions of the linker (e.g., HB and A) when subjected to certain metabolic, physiological, or cellular conditions that may initiate a cascade of fragmentation (e.g., after administration to a subject) for more than about 1 week (e.g., 1 week), more than about 1 month (e.g., 1 month), more than about 4 months (e.g., 4 months), more than about 6 months (e.g., 6 months), or more than about 1 year (e.g., 1 year). In certain embodiments, a slow-release linker comprises one or more amide bonds.

The conjugates hereof can be formulated with a quick-release linker or a slow-release linker as desired and/or suitable for the particular application. Generally, for imaging and radiotherapy applications, it can be generally desirable that the active agent and/or imaging agent is/are not readily released following administration to a subject. As such, it may be preferential for a conjugate for use in imaging or radiotherapy applications to comprise a slow-release linker, as opposed to a quick-release linker.

On the other hand, high and/or sustained kidney uptake can be a common feature of peptide-based pharmaceuticals, which can lead to reduced detection sensitivity for lesions adjacent to kidneys and renal toxicities as the kidneys are the main organs responsible for excreting these drugs from a body. There are various ways to reduce renal uptake and retention, such as co-administering positively charged amino acids (e.g., lysine) along with the drug. Another conventional method is to utilize unnatural amino acids (UAAs) such as D-amino acids as these are not as readily recognized by renal proteins as compared with natural amino acids. Additionally or alternatively, quick-release linkers can be incorporated into a conjugate that can be fragmented or cleaved in a kidney to prevent uptake.

A brush border is the name for the microvilli-covered surface of the pseudostratified columnar epithelium found in multiple locations of the body, the two main locations being the small intestine tract and the kidney. In the kidney, the brush border is useful in distinguishing the proximal tubule (which possesses the brush border) from the distal tubule (which does not). In a proximal tubule of a kidney, various brush border membrane (BBM) peptidases can bind to peptides and cleave them so the peptides can either be recycled or excreted. Several BBM peptidases (which are quick-release linkers) are known in the art including the tripeptide Met-Val-Lys, the dipeptide Gly-Lys, and the tripeptide Gly-Phe-Lys. The term “peptidase” refers to an enzyme which hydrolyzes peptide bonds between amino acids. Previous studies have tested these sequences as linkers between a targeting moiety and a bioactive agent and have confirmed inclusion of these linkers in a conjugate can expedite renal clearance.

In certain embodiments, L_{A} comprises a BBM peptidase linker. The BBM linker can be a quick-release linker. Insertion of a BBM linker between the ligand/targeting moiety (HB) and the imaging or therapeutic agent (A) of the conjugate can result in recognition and cleavage by renal brush border enzymes.

In certain embodiments, the BBM linker comprises a cleavable tripeptide Met-Val-Lys. In certain embodiments, the BBM linker can comprise a tripeptide Gly-Tyr-Lys. When a conjugate comprising a BBM linker is administered to a subject (e.g., systemically), the BBM linker can cleave and release the ligand/targeting moiety (HB) and the imaging or therapeutic agent (A) therefrom (e.g., under physiological conditions).

The BBM linker can, in certain embodiments, include one or more UAAs including, without limitation, one of more of a D-amino acid, citrulline, hydroxyproline, norleucine, 3-nitrotyrosine, nitroarginine, naphthylalanine, aminobutyric acid (Abu), 2,4-diaminobutyric acid (DAB), methionine sulfoxide, methionine sulfone, and the like.

It should be appreciated that physiological conditions resulting in a BBM linker breaking include standard chemical hydrolysis reactions that occur, for example, at physiological pH, or as a result of compartmentalization into a cellular organelle, such as an endosome having a lower pH than cytosolic pH. Illustratively, the BBM linkers described herein can undergo cleavage (e.g., quick-release) under certain metabolic, physiological, or cellular conditions that may initiate a cascade of fragmentation or bond cleavage (which may, for example, result in the release of one or more of the moieties connected through one or more portions of the linker (e.g., HB and A)).

The atoms used in forming L_{A} and L_{AB} can be combined in all chemically relevant ways, such as chains of carbon atoms forming alkylene groups, chains of carbon and oxygen atoms forming polyoxyalkylene groups, chains of carbon and nitrogen atoms forming polyamines, and others. In addition, the bonds connecting atoms in the chain can be either saturated or unsaturated, such that, for example, alkanes, alkenes, alkynes, cycloalkanes, arylenes, imides, and the like can be divalent radicals that are included in L_{A} and L_{AB}. In addition, the atoms forming the linker can be cyclized upon each other to form saturated or unsaturated divalent cyclic radicals in the linker.

In some embodiments, L_{A} and L_{AB} can comprise portions that are neutral under physiological conditions. In some embodiments, L_{A} and L_{AB} can comprise portions that can be protonated or deprotonated to carry one or more positive or one or more negative charges, respectively. In some embodiments, L_{A} and L_{AB} can comprise neutral portions and portions that can be protonated to carry one or more positive charges.

Examples of neutral portions include polyhydroxyl groups, such as sugars, carbohydrates, saccharides, inositols, and the like, and/or polyether groups, such as polyoxyalkylene groups, including polyoxyethylene, polyoxypropylene, and the like.

Examples of portions that can be protonated to carry one or more positive charges include amino groups, such as polyaminoalkylenes, including ethylene diamines, propylene diamines, butylene diamines and the like, and/or heterocycles, including pyrrolidines, piperidines, piperazines, and other amino groups, each of which can be optionally substituted. In certain embodiments, L_{A} and L_{AB} can comprise a positive portion comprising one or more lysine residues.

Examples of portions that can be deprotonated to carry one or more negative charges include carboxylic acids, such as aspartic acid, glutamic acid, and longer chain carboxylic acid groups, and sulfuric acid esters, such as alkyl esters of sulfuric acid.

Alternatively, or in addition to chain length, in some embodiments, L_{A} and L_{AB} have suitable substituents that can affect hydrophobicity or hydrophilicity. Thus, for example, L_{A} and L_{AB} can have a hydrophobic side chain group, such as an alkyl, cycloalkyl, aryl, arylalkyl, or like group, each of which is optionally substituted. If L_{A} and L_{AB} were to include one or more amino acids, L_{A} and L_{AB} can contain hydrophobic amino acid side chains, such as one or more amino acid side chains from Phe and Tyr, including substituted variants thereof, and analogs and derivatives of such side chains.

Either or both of L_{A} and L_{AB} can comprise a slow-release linker. In some embodiments, a slow-release linker comprises a backbone that is stable under physiological conditions (e.g., the backbone is not susceptible to hydrolysis (e.g., aqueous hydrolysis or enzymatic hydrolysis)). In some embodiments, a slow-release linker does not release any component to which it is conjugated. In some embodiments, the slow-release linker lacks a disulfide bond (e.g., S—S) or an ester in the backbone. In some embodiments, HB and A are connected by a backbone that is substantially stable for the entire duration of the composition's circulation (e.g., in vivo). The slow-release linker can comprise an alkyl(ene), anhydride, amide, ester, ether, amine, and/or thioether (e.g., thio-maleimide). Any slow-release linker can be used provided at least one bond that is not easily or quickly broken under physiological conditions is formed.

In some embodiments, a non-releasable linker comprises a linker that, at a neutral pH, for example, less than ten percent (10%) (e.g., less than 5%, less than 4%, less than 3%, less than 2%, less than 1%, less than 0.1%, less than 0.01%, or less than 0.001%) will hydrolyze in an aqueous (e.g., buffered (e.g., phosphate buffer)) solution within a period of time (e.g., 24 hours). In some embodiments, where a slow-release linker is employed, less than about ten percent (10%), and preferably less than five percent (5%) or none, of the conjugate administered releases A (e.g., in systemic circulation prior to uptake by the targeted cells/tissue). In some embodiments, within one (1) hour of administration, less than five percent (5%) of A is released from the conjugate while the compound is in systemic circulation.

L_{A} can comprise a quick-release linker. A quick-release linker can release the A from the conjugate relatively quickly, for example, when subjected to physiological conditions (e.g., when in serum circulation). Examples of quick-release linkers include, but are not limited to, ester, disulfide, and thiol. L_{A} can comprise a quick-release linker. A cleavable bond for a quick-release linker can be part of the backbone.

The linker L_{A} can comprise a biodegradable, pH-sensitive, self-immolative, peptidase-sensitive, or hydrolysable linker. Examples of these linkers include, but are not limited to, a β-glucuronide linker, a maleimide-based thiol linker, a cathepsin K-sensitive linker, a cathepsin B-sensitive linker, a matrix metalloproteinase-sensitive linker, and a BBM-cleavable linker. Quick-release groups also include photochemically cleavable groups. Examples of photochemically-cleavable groups include 2-(2-nitrophenyl)-ethan-2-ol groups and linkers containing o-nitrobenzyl, desyl, trans-o-cinnamoyl, m-nitrophenyl or benzylsulfonyl groups (see, for example, Dorman and Prestwich, Trends Biotech. 18: 64-77 (2000); Greene & Wuts, Protective Groups in Organic Synthesis, 2nd ed., John Wiley & Sons, New York (1991)). The linkers L_{A} and L_{AB} can be conjugated to the N- or C-terminus of HB, any monomer of a polymer thereof, or any substituent of any monomer of a polymer thereof.

Both quick-release and slow-release linkers may be engineered to optimize biodistribution, bioavailability, and PK/PD (e.g., of the A) and/or to increase uptake (e.g., of A) into the targeted tissue pursuant to methodologies commonly known in the art or hereinafter developed, such as through PEGylation and the like. In some embodiments, the linker is configured to avoid significant release of a pharmaceutically active amount of A in circulation prior to capture by a cell.

It is appreciated that the lability of the quick-release bond can be adjusted by including functional groups or fragments within the linker that are able to assist or facilitate such bond cleavage, also termed anchimeric assistance. The lability of the quick-release bond can also be adjusted by, for example, substitutional changes at or near the quick-release bond, such as including alpha branching adjacent to a quick-release disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like. In addition, it is appreciated that additional functional groups or fragments can be included within the linker that are able to assist or facilitate additional fragmentation of the compounds after bond breaking of the quick-release linker, when present.

L_{A} can comprise L₁ or L₂:

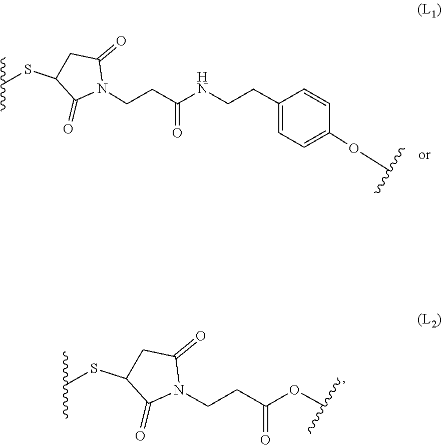 [SMILES: *Oc1ccc(CCNC(=O)CCN2C(=O)CC(S*)C2=O)cc1 ; *OC(=O)CCN1C(=O)CC(S*)C1=O]

in which

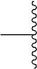

represents the point of attachment between L_{A} and A or L_{A} and HB.

Either or both of L_{A} and L_{AB} can comprise a spacer (e.g., be conjugated with and/or include a spacer). The spacer can be any suitable spacer. A spacer of L_{A} and L_{AB} can comprise hydrophilic, hydrophobic, amphipathic, non-peptidic, peptidic, and/or aromatic monomers. A length of a spacer can range from 1 to 30 (e.g., 1 to 30 carbon atoms, a PEG with 1-30 units, etc.). Examples of hydrophilic spacers include, but are not limited to, polyethylene glycol polymers and derivatives thereof. Examples of hydrophobic spacers include, but are not limited to, pure or mixed branched hydrocarbons, fluorocarbons, alkane, alkene, and/or alkyne polymers. Examples of amphipathic spacers include, but are not limited to, pure or mixed phospholipids and/or derivatives thereof. Examples of peptidic spacers include, but are not limited to, pure and mixed single, branched, L- or D-configurations, essential, nonessential, natural, and unnatural amino acids and derivatives thereof. Examples of aromatic spacers include, but are not limited to, pure and mixed repeated quinoids.

In some embodiments, the linker is formed via click chemistry/click chemistry-derived synthetic methods. Those of skill in the art understand that the terms “click chemistry” and “click chemistry-derived” generally refer to a class of small molecule reactions commonly used in conjugation, allowing the joining of substrates of choice with specific molecules. Click chemistry is not a single specific reaction but describes a way of generating products that follow examples in nature, which also generate substances by joining small modular units. In many applications click reactions join a biomolecule and a reporter molecule. Click chemistry is not limited to biological conditions; the concept of a “click” reaction has been used in pharmacological and various biomimetic applications. However, they have been made notably useful in the detection, localization and qualification of biomolecules.

Click reactions can occur in one pot, typically are not disturbed by water, can generate minimal byproducts, and are “spring-loaded”—characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (e.g., for in vivo systems).

A can be an active agent comprising a radio-imaging agent, a radio-sensitizing agent, a radio-protecting agent, or a radiotherapeutic agent. A can comprise a chelator (e.g., a chelating agent). In certain embodiments, the chelator (A) is covalently linked to the radical of the ligand/biological targeting moiety (HB) directly or via a linker (L_{A}). The chelator can comprise a functional group suitable for conjugation to the radical of the ligand/biological targeting moiety (HB) directly or via a linker (L_{A}). The term “functional group suitable for conjugation” means a functional group which will react with a corresponding functional group of HB or L_{A}, as desired, (for example, and without limitation, an amine, carboxyl or thiol group) to chemically link the chelator thereto.

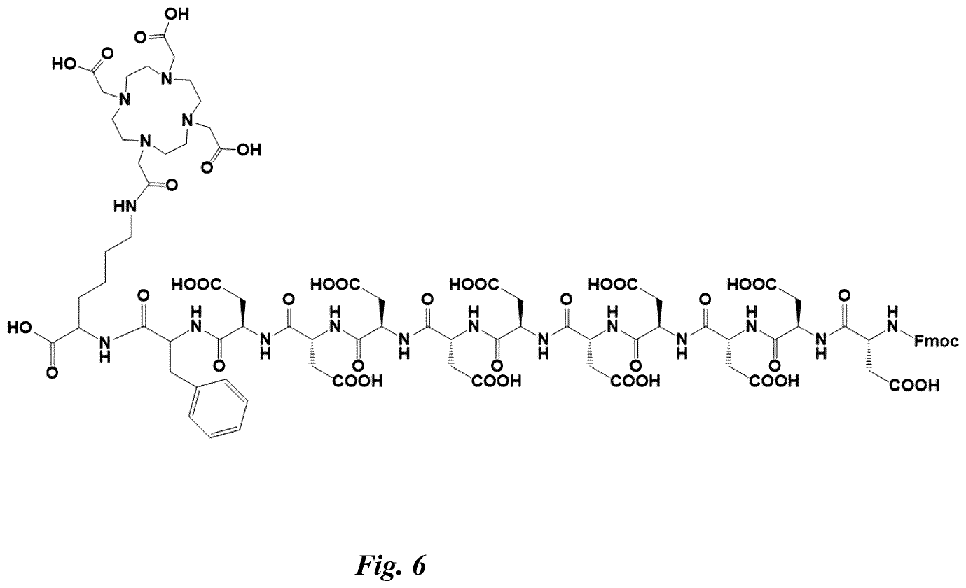

In certain embodiments, A comprises DOTA (1,4,7,10-tetraazacyclododecane-1,4,7,10-tetraacetic acid) or a derivative thereof (see, e.g., the conjugates shown in FIGS. 6, 7, 16, 20, 22, and 24). In certain embodiments, A comprises a chelator selected from the group consisting of DOTA or a derivative thereof; TETA (1,4,8,11-tetraazacyclotetradecane-1,4,8,11-tetraacetic acid) or a derivative thereof, HEHA (1,2,7,10,13-hexaazacyclooctadecane-1,4,7,10,13,16-hexaacetic acid) or a derivative thereof; PEPA (1,4,7,10,13-pentaazacyclopentadecane-N,N′,N″,N′″,N″″-pentaacetic acid) or a derivative thereof, SarAr (1-N-(4-Aminobenzyl)-3,6,10,13,16,19-hexaazabicyclo[6.6.6]-eicosane-1,8-diamine) or a derivative thereof; NOTA (1,4,7-triazacyclononane-1,4,7-triacetic acid) or a derivative thereof, NETA (4-[2-(bis-carboxymethylamino)-ethyl]-7-carboxymethyl-[1,4,7]triazonan-1-yl) acetic acid or a derivative thereof; TRAP (1,4,7-triazacyclononane-1,4,7-tris[methyl(2-carboxyethyl)phosphinic acid) or a derivative thereof; HBED (N,N0-bis(2-hydroxybenzyl)-ethylenediamine-N,N0-diacetic acid) or a derivative thereof, 2,3-HOPO (3-hydroxypyridin-2-one) or a derivative thereof; PCTA (3,6,9,15-tetraazabicyclo[9.3.1]-pentadeca-1(15),11,13-triene-3,6,9-triacetic acid) or a derivative thereof; PDTA (1,3-propylenediaminetetraacetate) or a derivative thereof, NTA (nitrilotriacetate) or a derivative thereof; EDDS (ethylenediaminedisuccinate) or a derivative thereof; EDTA (ethylene diamine tetraacetic acid) or a derivative thereof, MGDA (N-(1-carboxylatoethyl)iminodiacetate) or a derivative thereof, DFO (desferrioxamine) or a derivative thereof, DTPA (diethylenetriaminepentaacetic acid) or a derivative thereof, CDTA ((1,2-cyclohexylenedinitrilo)tetraacetic acid) or a derivative thereof, CPTA (1,4,8,11-tetraazacyclotetradecane derivative) or a derivative thereof; OCTAPA (N,N0-bis(6-carboxy-2-pyridylmethyl)-ethylenediamine-N,N0-diacetic acid) or a derivative thereof, H2-MACROPA (N,N′-bis[(6-carboxy-2-pyridipmethyl]-4,13-diaza-18-crown-6) or a derivative thereof; H₂dedpa (1,2-[[carboxy)-pyridin-2-yl]-methylamino]ethane or a derivative thereof, HEDP (hydroxyethylidene diphosphonate or etideronate) or a derivative thereof, HEBD (N,N′-bis(2-hydroxybenzyl) ethylenediamine-N,N′-diacetic acid) or a derivative thereof, HYNIC (6-Hydrazinopridine-3-carboxylic acid) or a derivative thereof; DMSA (meso-2,3-dimercaptosuccinic acid) or a derivative thereof, and EC20-head comprising β-1-diaminopropionic acid, Asp, and Cys.

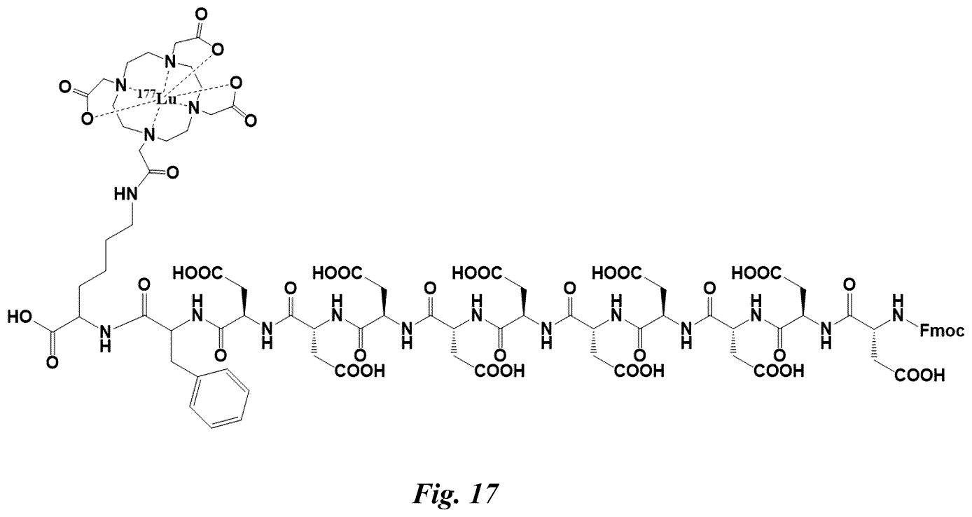

The chelator can chelate a radioisotope. In certain embodiments, the radioisotope is a therapeutic radioisotope, such as, for example, an α-emitting radioisotope (e.g., ²²⁵Ac) or a β-emitting radioisotope (e.g., ¹⁷⁷Lu or ⁹⁰Y). In certain embodiments, the conjugate comprises the ¹⁷⁷Lu-complexed DOTA conjugate shown in FIG. 17.

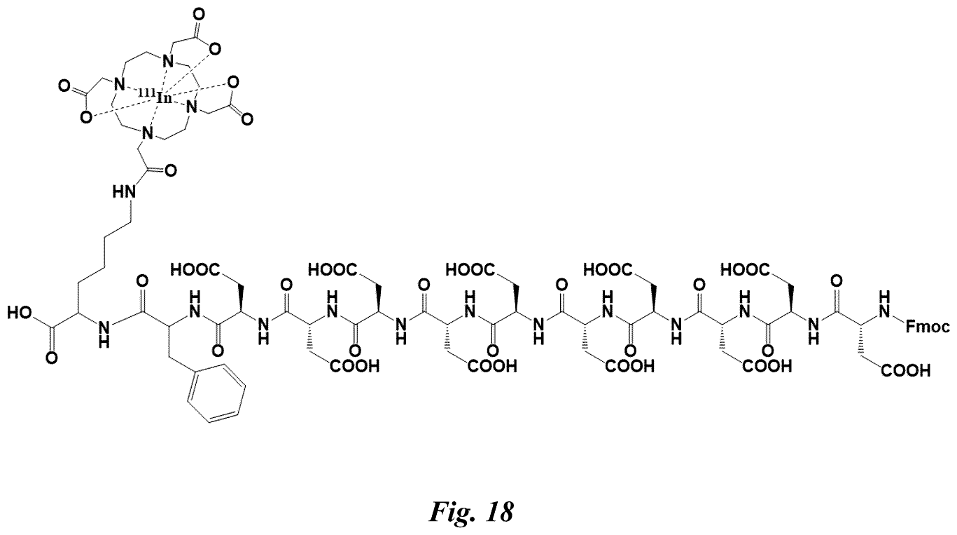

A of Formula I or Formula IA can comprise a chelator comprising an imaging radioisotope. In certain embodiments, the radioisotope comprises lutetium (Lu) or indium (In). In certain embodiments, the imaging radioisotope can be a γ-emitting radioisotope, such as ¹¹¹In or ⁶⁷Ga. In certain embodiments, the conjugate comprises the ¹¹¹In-complexed DOTA conjugate shown in FIG. 18. The chelator can chelate ¹⁸F, ⁴⁴Sc, ⁴⁷Sc, ⁵²Mn, ⁵⁵Co, ⁶⁴Cu, ⁶⁷Cu, ⁶⁷Ga, ⁶⁸Ga, ⁸⁶Y, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ab, ²²⁵Ac, or ²²⁷Th. In certain embodiments, the chelator is a positron emission tomography (PET) imaging radioisotope, such as ⁶⁸Ga, ¹¹C, ¹³N, ¹⁵O, and ¹⁸F. A can comprise a radioisotope selected from the group consisting of ¹⁴C, ³H, ³⁴S, ³²P, ¹²⁵I, and ¹³¹I. The radioactive agent A can be conjugated to the N- or C-terminus of HB or to any active group on repeating moieties of HB via linker L_{A}.

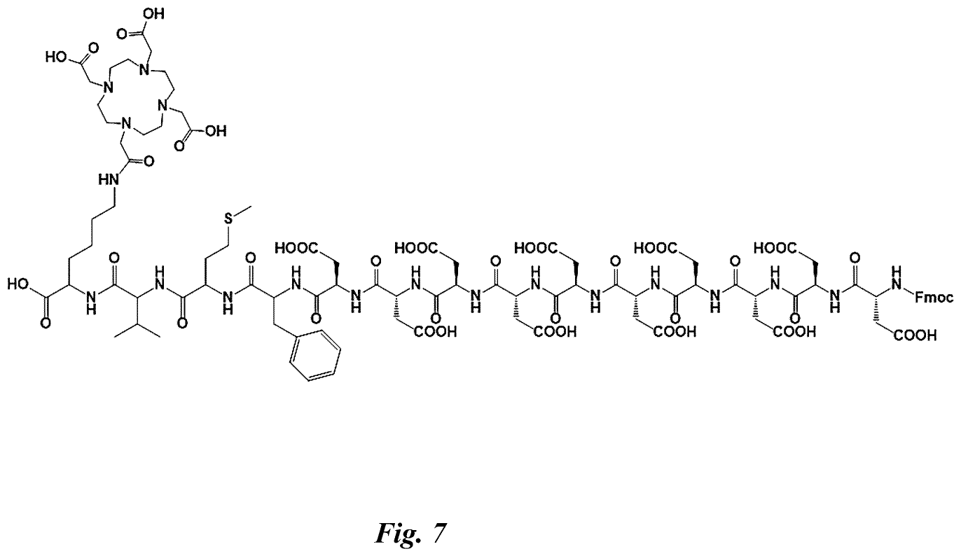

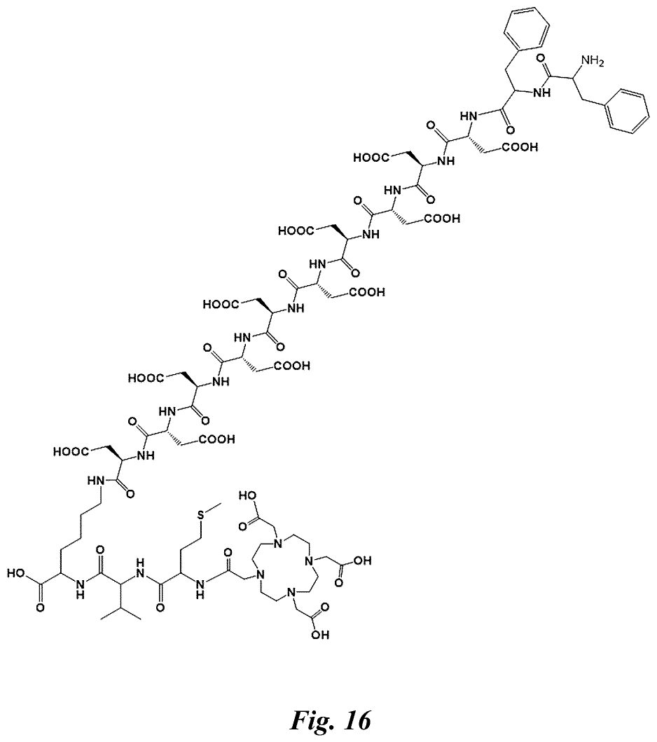

The conjugate can have the structure shown in FIG. 4. The conjugate can have the structure shown in FIG. 6. The conjugate can have the structure shown in FIG. 7. The conjugate can have the structure shown in FIG. 16.

The HB of the conjugate can comprise D-γ-carboxyglutamic acid, L-γ-carboxyglutamic acid, or a mixture of two or more of the foregoing or derivatives thereof, and the A can comprise DOTA or a near infrared dye, such as S0456. The conjugate can have one of the following structures:

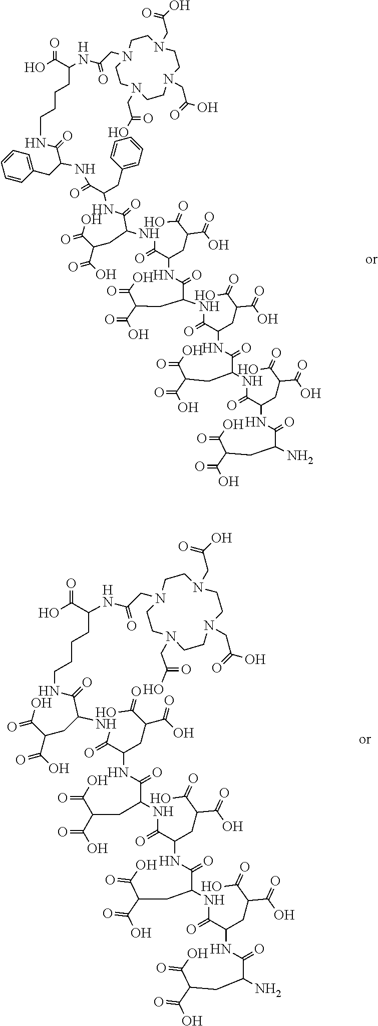

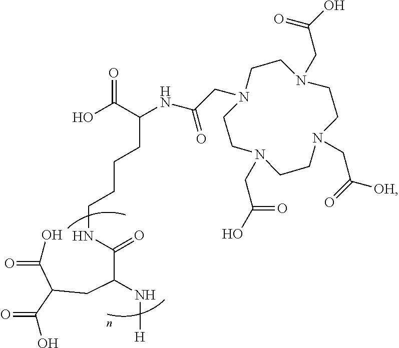

wherein n=1-50.

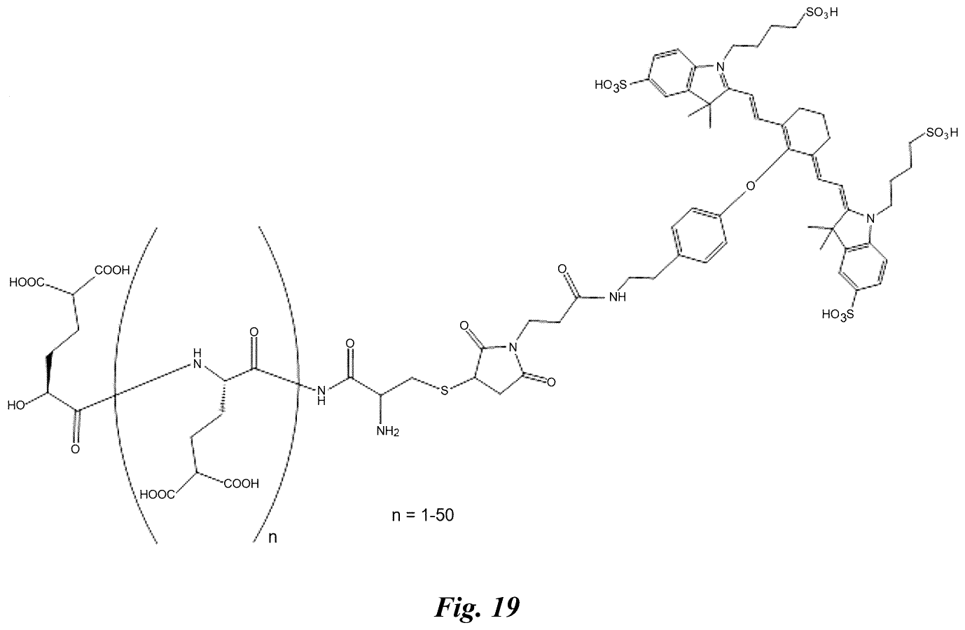

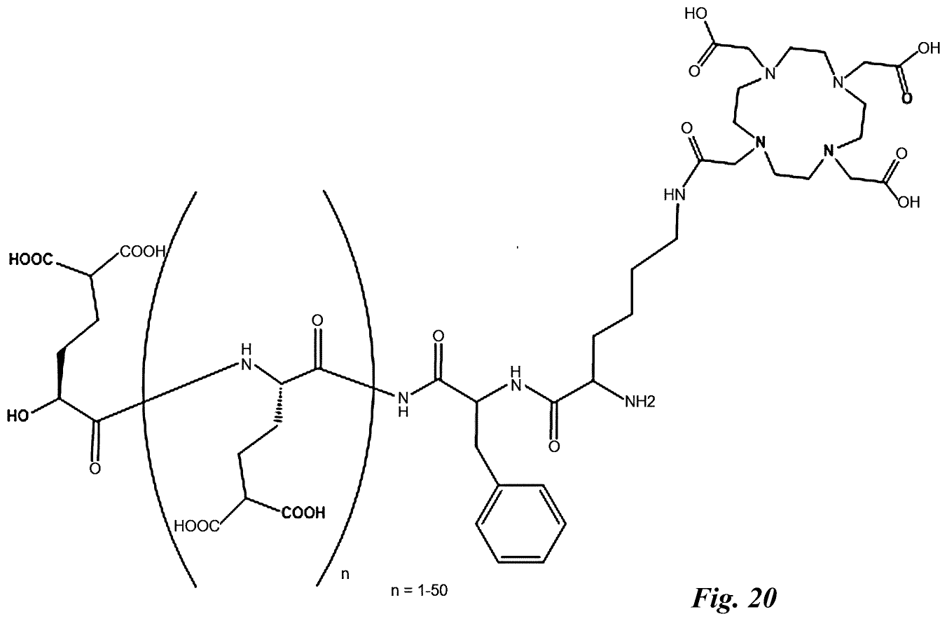

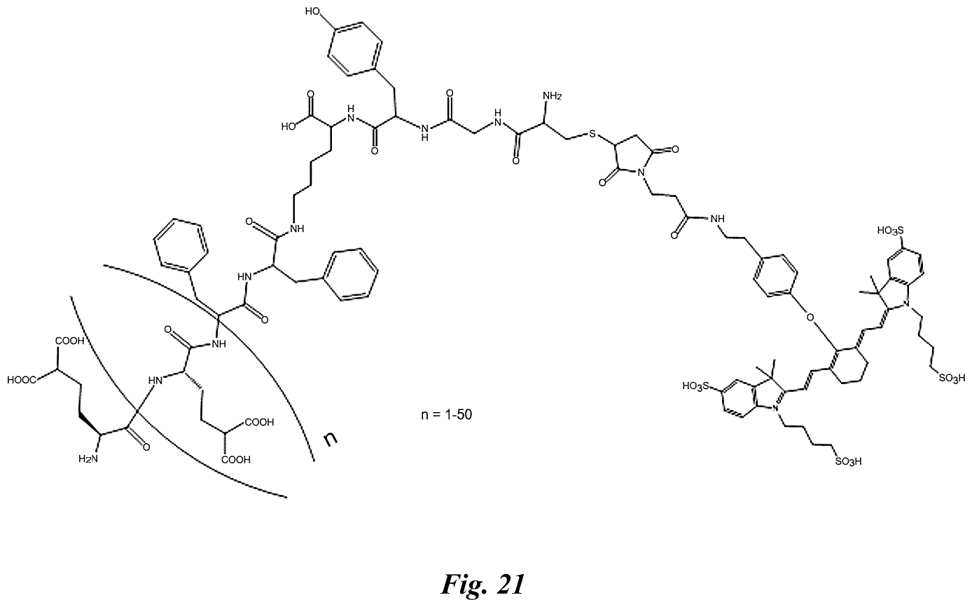

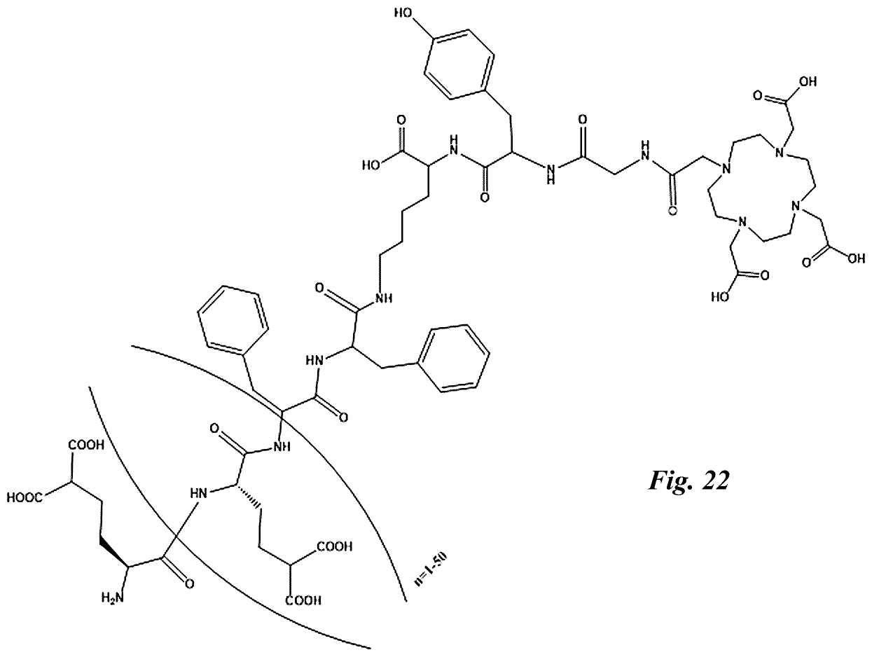

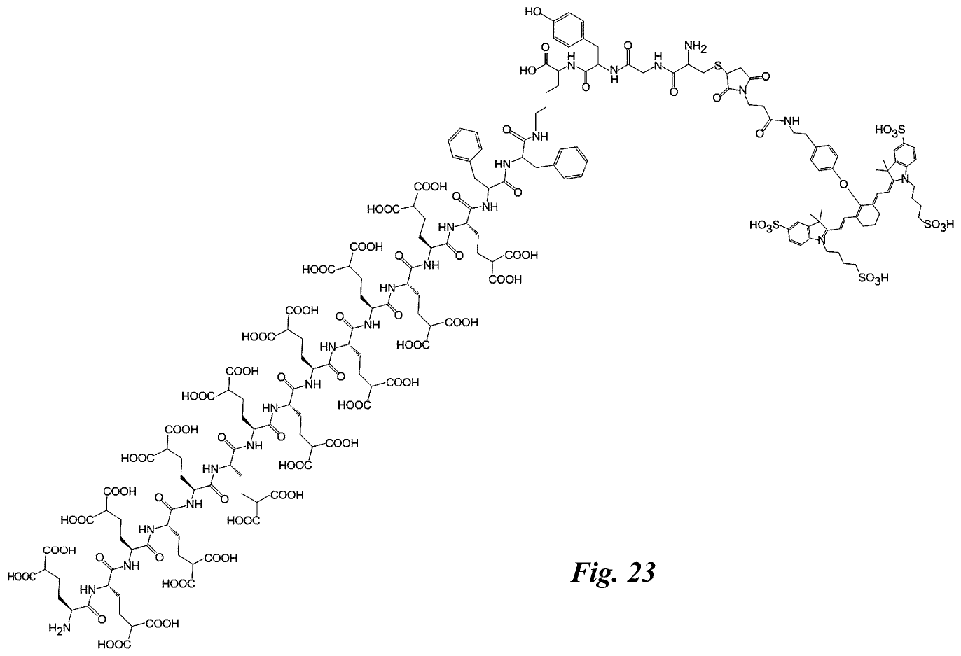

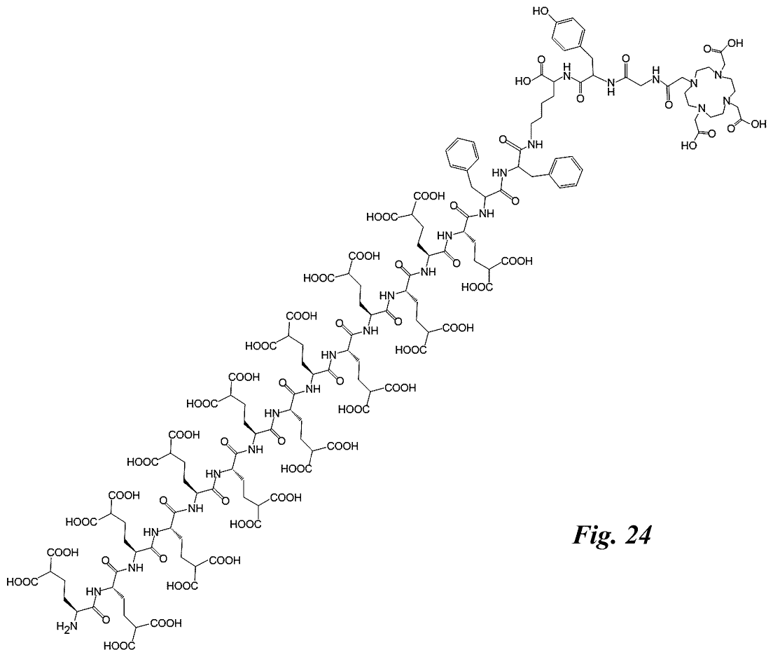

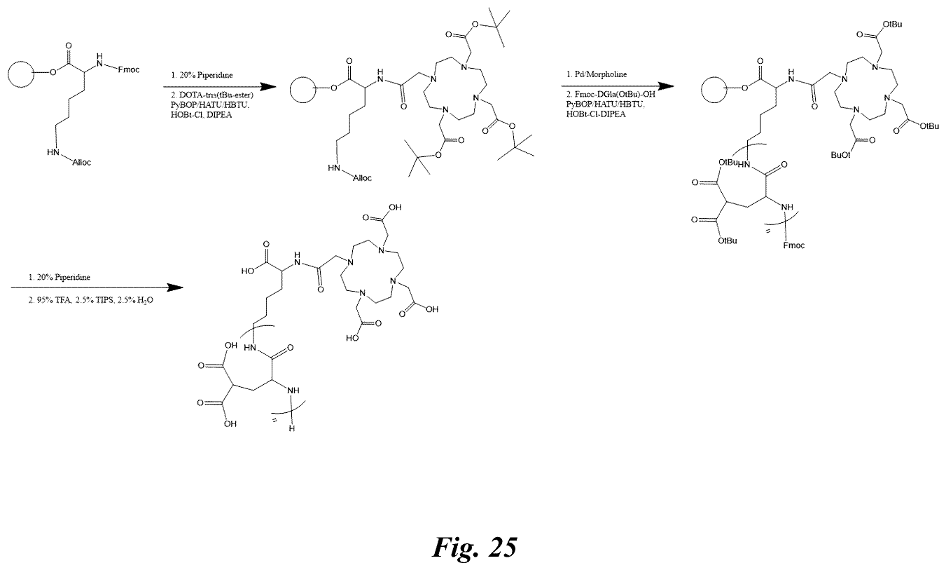

The conjugate can have the structure shown in FIG. 19. The conjugate can have the structure shown in FIG. 20. The conjugate can have the structure shown in FIG. 21. The conjugate can have the structure shown in FIG. 22. The conjugate can have the structure shown in FIG. 23. The conjugate can have the structure shown in FIG. 24.

The above conjugates can be synthesized using methods known in the art and exemplified herein. See, for example, Example 4.

The conjugate can be presented as a pharmaceutically acceptable salt. Examples of acceptable salts include, without limitation, alkali metal (e.g., sodium, potassium, or lithium) or alkaline earth metal (e.g., calcium) salts; however, any salt that is generally non-toxic and effective when administered to the subject being treated is acceptable. Similarly, “pharmaceutically acceptable salt” refers to those salts with counter ions, which can be used in pharmaceuticals. Such salts can include, without limitation, (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or (2) salts formed when an acidic proton present in the parent compound either is replaced by a metal ion, e.g., an alkali metal ion, an alkaline earth ion, or an aluminum ion, or coordinated with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Acceptable salts can be obtained using standard procedures known in the art, including (without limitation) reacting a sufficiently acidic compound with a suitable base affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds can be formed from bases that form non-toxic salts. Illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemi-salts of acids and bases, such as hemi-sulphate and hemi-calcium salts, also can be formed.

The conjugates hereof can be “deuterated,” meaning one or more hydrogen atoms can be replaced with deuterium. As deuterium and hydrogen have nearly the same physical properties, deuterium substitution is the smallest structural change that can be made. Deuteration is well known to those of ordinary skill in the art.

The conjugates, in some embodiments, can contain one or more asymmetric centers and thus give rise to enantiomers, diastereomers, and other stereoisomeric forms that are defined, in terms of absolute stereochemistry, as (R)- or (S)-. In certain embodiments, the conjugate is of R-configuration. In certain embodiments, the conjugate is of S-configuration. Unless stated otherwise, it is intended that all stereoisomeric forms of the conjugates are contemplated. When the conjugates contain alkene double bonds, and unless specified otherwise, it is intended that both E and Z geometric isomers (e.g., cis or trans) are included. In certain embodiments, HB and A are arranged in a relative cis orientation. In certain embodiments, HB and A are arranged in a relative trans orientation. Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

### Pharmaceutical Compositions

In view of the above, further provided is a pharmaceutical composition comprising any of the conjugates. In certain embodiments, provided herein is a pharmaceutical composition comprising a conjugate (e.g., a conjugate of Formula (I) or Formula (IA)) and one or more pharmaceutically acceptable carriers or excipients. The term “composition” generally refers to any product comprising more than one ingredient, including the conjugate. The compositions can be prepared from isolated conjugates or from salts, solutions, hydrates, solvates, and other forms of the conjugates.

In certain embodiments, a conjugate of the composition comprises the conjugate of Formula I (or the pharmaceutically acceptable salt thereof):

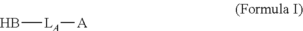

wherein HB is a radical of an (e.g., straight-chain or branched-chain) anionic, acidic, and/or electrolytic ligand (e.g., that binds hydroxyapatite); A is an active agent comprising a radio-imaging agent, a radio-sensitizing agent, a radio-protecting agent, or a radiotherapeutic agent; and L_{A} is a linker that binds HB and A (as described herein). In certain embodiments, L_{A} is optional and, where not included, HB is conjugated directly to A.

In certain embodiments, a conjugate of the composition comprises the conjugate of Formula I (or the pharmaceutically acceptable salt thereof):

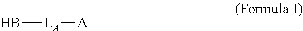

wherein HB is a radical of a D-γ-carboxyglutamic acid ligand, L-γ-carboxyglutamic acid ligand, or a mixture of the foregoing or derivatives thereof (e.g., that binds hydroxyapatite); A is an active agent comprising a radio-imaging agent, a radio-sensitizing agent, a radio-protecting agent, or a radiotherapeutic agent; and L_{A} is a linker that binds HB and A (as described herein). In certain embodiments, L_{A} is optional and, where not included, HB is conjugated directly to A.

In certain embodiments, L_{A} is optional and, where not included, HB is conjugated directly to A. For example, in certain embodiments, a conjugate comprises Formula IA:

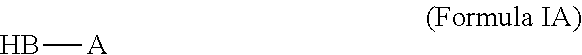

or a pharmaceutically acceptable salt thereof, wherein HB is a radical of an anionic, acidic, and/or electrolytic ligand (e.g., that binds hydroxyapatite); and A is an active agent comprising a radio-imaging agent, a radio-sensitizing agent, a radio-protecting agent, or a radiotherapeutic agent. In certain embodiments, HB functions as a targeting moiety for the conjugate.

In certain embodiments, the ligand (e.g., HB) can comprise from one to about 50 (e.g., 1-50) anionic, acidic, and/or electrolytic moieties. In certain embodiments, the ligand (e.g., HB) can comprise an amino acid or a derivative thereof. In certain embodiments, the ligand (e.g., HB) can comprise a L-configuration. In certain embodiments, the ligand (e.g., HB) can comprise a D-configuration. In certain embodiments, the ligand (e.g., HB) can further comprise one or more hydrophobic moieties. The ligand (e.g., HB) can further comprise a serum AB conjugated directly to HB or conjugated via to a linker L_{AB} (AB-L_{AB}) to HB (e.g., wherein AB-L_{AB} is conjugated to HB via L_{AB}. In certain embodiments, one or more peptide bonds of HB and/or A are arranged in a relative cis orientation. In certain embodiments, one or more peptide bonds of HB and/or A are arranged in relative trans orientation. In certain embodiments, the conjugate is of R-configuration. In certain embodiments, the conjugate is of S-configuration.

In certain embodiments, the ligand (e.g., HB) comprises a radical of a straight-chain polyanion of formula X-A₁, X-A₂ X-A₃, or X-A₄:

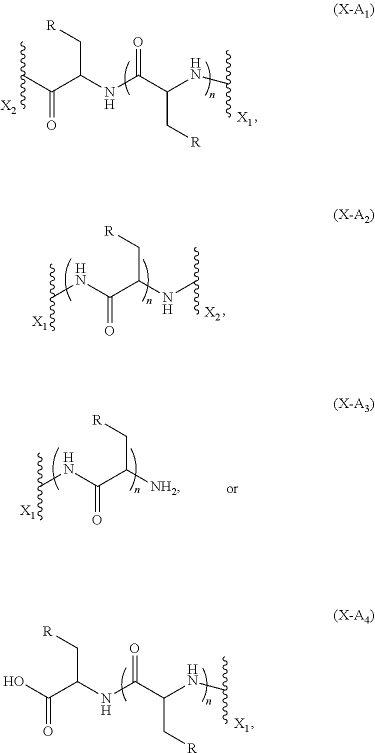

wherein:
R is COOH for L- or D-aspartic acid, CH₂COOH for L- or D-glutamic acid, HOOC—CH₂—CH(COOH)—CH(NH₂)—COOH or D- or L-γ-carboxyglutamic acid, or CH₂CH₂COOH for an unnatural, acidic amino acid;
n=1-50; and

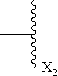

represents the point of attachment to AB or L_{AB}.

HB can comprise a radical of a branched-chain polyanion of formula X-A₅, X-A₆, X-A₇, or X-A₈.

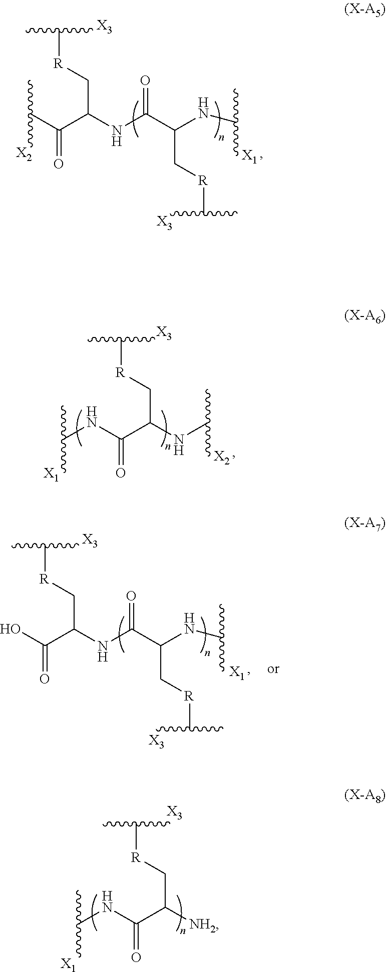

wherein:
R is COO— for L- or D-aspartic acid, CH₂COO— for L- or D-glutamic acid, HOOC—CH₂—CH(COOH)—CH(NH₂)—COOH or D- or L-γ-carboxyglutamic acid, or CH₂CH₂COO— for an unnatural, acidic amino acid;
n=1-50;

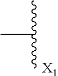

represents the point of attachment to L_{A}; and,
when HB further comprises AB or AB-L_{AB},

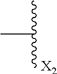

represents the point of attachment to AB or L_{AB}, and

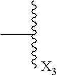

represents the point of attachment to hydrogen or an HB.

In certain embodiments, HB does not (may not) comprise an albumin binder (e.g., formulae X-A₃, X-A₄, X-A₇ and X-A₈). Linkers, represented by L_{A} and L_{AB}, can be any suitable linkers (e.g., a quick-release or slow-release linker).

A can be an active agent comprising a radio-imaging agent, a radio-sensitizing agent, a radio-protecting agent, or a radiotherapeutic agent. For example, A can comprise a chelator (e.g., any suitable chelator including, without limitation, those specified herein in connection with the conjugates). A can comprise a radioisotope. A can comprise a PET imaging radioisotope. The active agent of A can be conjugated to the N- or C-terminus of HB or to any active group on repeating moieties of HB via a linker (e.g., L_{A}).

Certain functional groups, such as the hydroxy, amino, and like groups can form complexes with water and/or various solvents, in the various physical forms of the conjugate.

The compositions can be prepared from various amorphous, non-amorphous, partially crystalline, crystalline, and/or other morphological forms of the conjugates, and the compositions can be prepared from various hydrates and/or solvates of the conjugates. Accordingly, such pharmaceutical compositions can include each of, or any combination of, or individual forms of, the various morphological forms and/or solvate or hydrate forms of the conjugates.

The pharmaceutical composition can comprise one or more pharmaceutically acceptable carriers, adjuvants, diluents, excipients, and/or vehicles (e.g., conventional nontoxic pharmaceutically acceptable carriers, adjuvants, and vehicles), and combinations thereof. Any pharmaceutically acceptable carriers and excipients as known in the art can be used. Examples include, but are not limited to, an excipient, a color additive, a preservative, and a stabilizer. More specific examples include crystal cellulose, calcium carmellose, sodium carmellose, hydropropylcellulose, hydroxypropylmethylcellulose, ethylcellulose, and magnesium stearate.

Solutions of the active conjugate or pharmaceutical composition can be aqueous, optionally mixed with a nontoxic surfactant, and/or can contain carriers or excipients, such as salts, carbohydrates and buffering agents (preferably at a pH of from 3 to 9), but, for some applications, they can be more suitably formulated as a sterile non-aqueous solution or as a dried form to be used in conjunction with a suitable vehicle, such as sterile, pyrogen-free water, or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical composition can further comprise a radiosensitizer, a radioprotector, an immunotherapeutic agent, a chemotherapeutic agent, an anti-cancer agent, and/or a hormone therapeutic agent. Alternatively, the conjugate (or pharmaceutical composition comprising same) can be administered simultaneously or sequentially, in either order, with a radiosensitizer, a radioprotector, an immunotherapeutic agent, an anti-cancer agent, a chemotherapeutic agent, and/or a hormone therapeutic agent (or a pharmaceutical composition comprising any of the foregoing).

The radio-sensitizer can be any suitable radio-sensitizer. Examples of suitable radio-sensitizers include, but are not limited to, a peptide or protein, such as HER3-ADC, SYM004, cetuximab, nimotuzumab, AMG102, C-reactive peptide, HSP, paraoxonase-2, and ECI301; a nanomaterial, such as gold (Au), GSH-modified Au, PEG-modified Au, silver (Ag), PEG-modified Ag, PVP-modified Ag, gadolinium (Gd), PEG-modified Gd, DTPA-modified Gd, hafnium (Hf), tantalum (Ta), tungsten (W), bismuth (Bi), and platinum (Pt); apaziquone, curcumin, misonidazole, tirapazamine, AQ4N, paclitaxel, fluorouracil, cisplatin, dihydroartemisinin, resveratrol, etanidazole, porfiromycin, TH-302, mitomycin C, nelfinavir, docetaxel, hydrogen peroxide, RRx-001, gemcitabine, or papaverine hydrochloride. See, e.g., Gong et al., Int J Nanomedicine 16: 1083-1102 (2021).

The radio-protector can be any suitable radio-protector. A nonlimiting example of a suitable radioprotector is amifostine. See, e.g., Gong et al. (2021), supra.

The immunotherapeutic agent can be any suitable immunotherapeutic drug. Examples of suitable immunotherapeutic drugs include, but are not limited to, a transforming growth factor beta (TGF-β) inhibitor, such as R268712, or programmed death-ligand 1 (PD-L1) inhibitor, such as Keytruda.

The anti-cancer agent can be any suitable anti-cancer drug. Examples of suitable anti-cancer drugs include, but are not limited to, a kinase inhibitor, such as dasatinib. In some embodiments, A is not dasatinib, such as when HB comprises at least 4 amino acids (e.g., 4-20 amino acids).

The chemotherapeutic drug can be any suitable chemotherapeutic drug. Examples of suitable chemotherapeutic drugs include, but are not limited to, an anthracycline, such as doxorubicin, taxane, such as docetaxel, cyclophosphamide, such as Cytoxan, or 5-fluoro-uracil.

The hormone or hormone-related therapeutic agent can be any suitable hormone or hormone-related therapeutic agent. Examples include, but are not limited to, a hormone-production inhibitor, such as Zoladex or letrozole.

The conjugates can be formulated as pharmaceutical compositions and administered to a mammalian host, such as a human patient, in a variety of forms adapted to the chosen route of administration. The pharmaceutical compositions can be formulated, e.g., for a given route of administration, and manufactured in accordance with methods in the art and described, for example, in Remington, The Science and Practice of Pharmacy, 22^{nd} edition (2012). The composition can be an infusion or an injectable composition, such as a composition that can be injected subcutaneously or intravenously.

The pharmaceutical composition can be administered to a mammalian host, such as a human patient, in a variety of forms adapted to the chosen route of administration. In certain embodiments, the pharmaceutical composition is formulated to be administered subcutaneously. In certain embodiments, the pharmaceutical composition is formulated to be administered orally. In certain embodiments, the pharmaceutical composition is formulated to be administered intramuscularly, intravenously, intraarterially, intraperitoneally, or as any other art-recognized route of parenteral administration.

In certain embodiments, the pharmaceutical composition is systemically administered in combination with a pharmaceutically acceptable vehicle. The percentages of the components of the compositions and preparations can vary and can be between about 1 to about 99% weight of the active ingredient(s) (e.g., the conjugate) and a binder, an excipient, a disintegrating agent, a lubricant, and/or a sweetening agent (as are known in the art). The amount of active conjugate in such therapeutically useful compositions is such that an effective dosage level can be obtained (e.g., in the serum or targeted tissue).

Illustrative means of parenteral administration include needle (including microneedle) injectors, needle-free injectors and infusion techniques, as well as any other means of parenteral administration recognized in the art. Parenteral formulations are typically aqueous solutions, which can contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, can readily be accomplished using standard pharmaceutical techniques well-known to those skilled in the art.

The pharmaceutical dosage forms suitable for administration can include sterile aqueous solutions or dispersions or sterile powders comprising the active ingredients that are adapted for the extemporaneous preparation of sterile injectable or infusible solutions or dispersions, optionally encapsulated in liposomes, nanocrystals, or polymeric nanoparticles. In all cases, the ultimate dosage form should be sterile, fluid, and stable under the conditions of manufacture and storage. The liquid carrier or vehicle can be a solvent or liquid dispersion medium comprising, for example and without limitation, water, electrolytes, sugars, ethanol, a polyol (e.g., glycerol, propylene glycol, liquid polyethylene glycols, and the like), vegetable oils, nontoxic glyceryl esters, and/or suitable mixtures thereof. In at least one embodiment, the desired fluidity can be maintained by the formation of liposomes, by the maintenance of the required particle size in the case of dispersions or by the use of surfactants.

Sterile injectable solutions can be prepared by incorporating the pharmaceutical compositions in the required amount of the appropriate solvent with one or more of the other ingredients set forth above, as required, followed by filter sterilization. In the case of sterile powders for the preparation of sterile injectable solutions, vacuum-drying and freeze-drying techniques can be employed, which can yield a powder of the active ingredient plus any additional desired ingredient present in the previously sterile-filtered solutions.

### Methods

Further provided is a method of imaging and/or treating a bone in a subject. The method comprises administering to the subject an effective amount of (i) a conjugate of Formula I, Formula IA, or a pharmaceutically acceptable salt of Formula I or Formula IA, (ii) a first pharmaceutical composition comprising a conjugate of Formula I, a conjugate of Formula IA, or a pharmaceutically acceptable salt of Formula I or IA and, optionally, wherein the first pharmaceutical composition further comprises a radiosensitizer, radioprotector, immunotherapeutic agent, chemotherapeutic agent, anti-cancer drug, or hormone therapeutic agent, and a first pharmaceutically acceptable carrier or excipient. In certain embodiments of the method, HB of Formula I comprises a radical of a D-γ-carboxyglutamic acid ligand, L-γ-carboxyglutamic acid ligand, or a mixture of the foregoing or derivatives thereof. In certain embodiments, HB comprises 7-20 D-γ-carboxyglutamic acid residues.

Additionally, as desired, the method can further comprise administering (i) or (ii) to the subject alone or in further combination with administration of an active agent or a second pharmaceutical composition comprising the active agent and a second pharmaceutically acceptable carrier or excipient. The active agent can be, for example, a radiosensitizer, radioprotectant (e.g., Lys), immunotherapeutic agent, chemotherapeutic agent, anti-cancer agent, or hormone therapeutic agent.

As used herein, the term “administering” and its variants include all means of introducing the compound(s) and compositions described herein to the subject, including, without limitation, oral (p.o.), intravenous (i.v.), intramuscular (i.m.), subcutaneous (s.c.), transdermal, via inhalation (e.g., intranasal (i.n.)), buccally, intraocularly, sublingually, vaginally, rectally, and the like.

As used herein, “effective amount” refers to an amount that is sufficient to achieve the desired result or to have an effect on an undesired condition. For example, an effective amount can refer to an amount that is sufficient to achieve the desired therapeutic result or to have an effect on symptoms. The specific effective dose level (e.g., amount) for any subject will depend upon a variety of factors including the disorder/condition being treated and the severity of the disorder/condition (e.g., the type, location, and severity of a fracture, bone injury, or cancer); the specific composition(s) and/or conjugate employed (i.e., the potency thereof); the age, body weight, general health, sex and diet of the subject; the response of the subject; the time of administration; the route of administration; the rate of excretion of the specific conjugate(s) employed; the duration of the treatment; drugs/active agents used in combination or coincidental with the specific conjugate employed and like factors that are well known in the medical arts. For example, it is well within the skill of the art to start doses of a conjugate/composition at levels lower than those required to achieve the desired therapeutic effect and to gradually increase the dosage until the desired effect is achieved. If desired, the effective amount can be divided into multiple doses for purposes of administration. Consequently, single dose conjugates/compositions can contain such amounts or submultiples thereof to make up the daily dose. The dosage can be adjusted by the individual physician in the event of any contraindications. Additionally, pharmacogenomic (the effect of genotype on the pharmacokinetic, pharmacodynamic or efficacy profile of the antigen or composition) information about a particular patient can affect the dosage used to achieve an effective amount.

Depending upon the route of administration, a wide range of permissible dosages is contemplated. For example, the effective amount of the conjugate and/or pharmaceutical composition can range from about 0.1 μg/kg/day, such as 0.5 μg/kg/day, 0.7 μg/kg/day, or 0.01 mg/kg/day up to about 1,000 mg/kg/day. Intravenous doses can be several orders of magnitude lower.

Where the conjugate and/or first pharmaceutical composition is administered with an active agent or second pharmaceutical composition comprising the active agent, the conjugate/first pharmaceutical composition and the active agent/second pharmaceutical composition can be administered to the subject simultaneously or sequentially, in either order and using any delivery modality. For example, and without limitation, the conjugate/first pharmaceutical composition can be administered intravenously (e.g., as an i.v. solution) and the active agent/second pharmaceutical composition can be administered subcutaneously. In other embodiments, both the conjugate/first pharmaceutical composition and the active agent/second pharmaceutical composition are administered intravenously. The use of a pharmaceutical composition comprising more than one drug/active agent is within the scope of this disclosure.

The conjugate and any other active agents administered therewith (or sequentially therewith) can be administered in therapeutically effective dosages to obtain clinically acceptable results, e.g., reduction or elimination of symptoms or of the tumor. Thus, the conjugate and any active agent(s) can be administered concurrently or consecutively in a treatment protocol. The administration of any active agents can be made according to treatment protocols already known in the art.

Those skilled in the art will appreciate that treatment protocols can be varied according to the needs of the subject. Thus, the combination of conjugates and other compounds (drugs) used in the methods hereof can be administered in variations of the protocols described herein. For example, the conjugates and/or active agents can be administered discontinuously rather than continuously during the treatment cycle.

The conjugate of Formula I (or the pharmaceutically acceptable salt thereof) can be any of the conjugates described herein. In certain embodiments, the conjugate or pharmaceutically acceptable salt thereof has the formula:

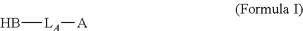

wherein HB is a radical of a (e.g., straight-chain or branched-chain) anionic, acidic, and/or electrolytic ligand (e.g., that binds hydroxyapatite); A is an active agent comprising a radio-imaging agent, a radio-sensitizing agent, a radio-protecting agent, or a radiotherapeutic agent; and L_{A} is a linker that binds HB and A, or is absent.

In certain embodiments, L_{A} is optional and, where not included, HB is conjugated directly to A. For example, in certain embodiments, a conjugate comprises Formula IA:

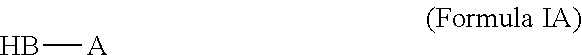

or a pharmaceutically acceptable salt thereof, wherein HB is a radical of an anionic, acidic, and/or electrolytic ligand (e.g., that binds hydroxyapatite); and A is an active agent comprising a radio-imaging agent, a radio-sensitizing agent, a radio-protecting agent, or a radiotherapeutic agent.

In certain embodiments, the ligand (e.g., HB) comprises from one to about 50 (e.g., 1-50) moieties. In certain embodiments, the ligand (e.g., HB) comprises an amino acid or a derivative thereof (e.g., of L- or D-configuration). In certain embodiments, the ligand (e.g., HB) further comprises one or more hydrophobic moieties. In certain embodiments, the ligand of HB comprises D-γ-carboxyglutamic acid moieties, L-γ-carboxyglutamic acid moieties, or a mixture of the foregoing or derivatives thereof. The ligand (e.g., HB) can further comprise a serum AB conjugated directly to HB or conjugated via to a linker L_{AB} (AB-L_{AB}) to HB (e.g., wherein AB-L_{AB} is conjugated to HB via L_{AB}).

In certain embodiments, the ligand (e.g., HB) comprises a radical of a straight-chain polyanion of formula X-A₁, X-A₂ X-A₃, or X-A₄:

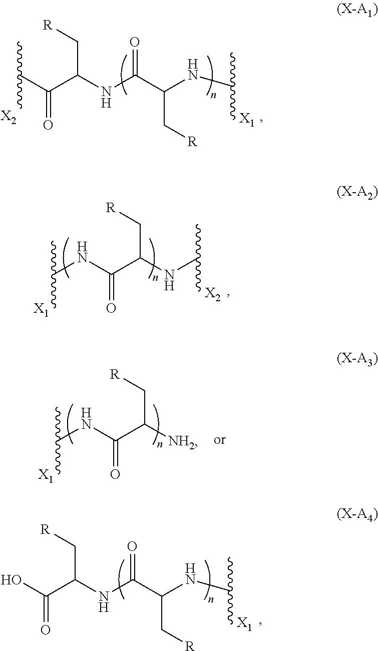

wherein:
R is COOH for L- or D-aspartic acid, CH₂COOH for L- or D-glutamic acid, HOOC—CH₂—CH(COOH)—CH(NH₂)—COOH or D- or L-γ-carboxyglutamic acid, or CH₂CH₂COOH for an unnatural, acidic amino acid;
n=1-50; and

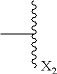

represents the point of attachment to AB or L_{AB}.

HB can comprise a radical of a branched-chain polyanion of formula X-A₅, X-A₆, X-A₇, or X-A₈.

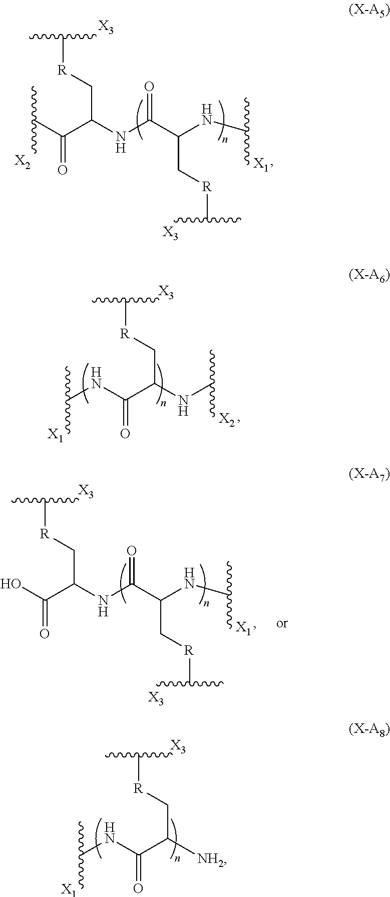

wherein:
R is COO— for L- or D-aspartic acid, CH₂COO— for L- or D-glutamic acid, HOOC—CH₂—CH(COOH)—CH(NH₂)—COOH or D- or L-γ-carboxyglutamic acid, or CH₂CH₂COO— for an unnatural, acidic amino acid;
n=1-50;

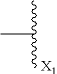

represents the point of attachment to L_{A}; and,
when HB further comprises AB or AB-L_{AB},

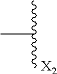

represents the point of attachment to AB or L_{AB}, and

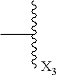

represents the point of attachment to hydrogen or an HB.

In certain embodiments, HB does not (may not) comprise an albumin binder (e.g., formulae X-A₃, X-A₄, X-A₇ and X-A₈). Linkers, represented by L_{A} and L_{AB}, can be any suitable linkers (e.g., a quick-release or slow-release linker).

A of Formula I or Formula IA can be an active agent comprising a radio-imaging agent, a radio-sensitizing agent, a radio-protecting agent, or a radiotherapeutic agent. For example, A comprises a chelator (e.g., any suitable chelator including, without limitation, those specified herein in connection with the conjugates). A can comprise a radioisotope. A can comprise a PET imaging radioisotope. The active agent of A can be conjugated to the N- or C-terminus of HB or to any active group on repeating moieties of HB via a linker (e.g., L_{A}).

In certain embodiments, the HB and A of the conjugate are arranged in a relative cis orientation. In certain embodiments, the HB and A of the conjugate are arranged in a relative trans orientation. In certain embodiments, the conjugate is of R-configuration. In certain embodiments, the conjugate is of S-configuration.

The method can further comprise the simultaneous or sequential administration, in either order, of an effective amount of an active agent that is a free radiosensitizer, radioprotector, immunotherapeutic agent, chemotherapeutic agent, anti-cancer drug, or hormone therapeutic agent, or a pharmaceutical composition (e.g., the second pharmaceutical composition) comprising same and a pharmaceutically acceptable carrier or excipient. In certain embodiments, the active agent comprises a radioprotectant. In certain embodiments, the radioprotectant active agent comprises Lys.

The subject can have cancer, such as primary bone cancer, e.g., osteosarcoma, chondrosarcoma, Ewing sarcoma, or chordoma, or secondary bone cancer, e.g., metastatic breast cancer, prostate cancer, multiple myeloma, thyroid cancer, lung cancer, kidney cancer, ovarian cancer, colon cancer, or melanoma.

In certain embodiments, the method can further comprise imaging the bone of the subject and/or a population of cancer cells in the subject (e.g., following administration of the conjugate, a first pharmaceutical composition comprising the conjugate, or a first pharmaceutical composition comprising the conjugate and a radiosensitizer, a radioprotector, an immunotherapeutic agent, a chemotherapeutic agent, an anti-cancer agent, and/or a hormone therapeutic agent). Imaging can be performed through any now known or hereinafter developed imaging techniques relevant to the medical arts. In certain embodiments, the imaging can be performed through a hybrid scanning, utilizing a functional imaging modality such as a single photon emission computer tomography (SPECT) or PET in combination with computed tomography (CT) and/or magnetic resonance imaging (MRI) techniques, and combinations thereof. Ultrasound imaging can also be used. As the conjugates hereof can be used to label the bone cancer cells (by binding, for example, hydroxyapatite), the methods hereof can be used to visualize, characterize, monitor and facilitate treatment of a bone cancer or other disease.

When the bone in the subject is imaged (either in connection with the method or separate therefrom), the method can further comprise diagnosing whether the subject has cancer.

When the subject has been treated for cancer and the bone in the subject is imaged, the method can further comprise assessing or monitoring the efficacy of treatment. For example, the conjugates and/or pharmaceutical compositions can be used to monitor tumor or lesion growth and proliferation quantitatively in vivo. In certain embodiments, a method of monitoring a progression of a cancer (e.g., a bone cancer) in a subject is provided, comprising administering a conjugate, a pharmaceutically acceptable salt thereof, or a pharmaceutical composition comprising the conjugate or a pharmaceutical acceptable salt thereof to a subject. Such method can further comprising imaging the cancer (and/or bone) of the subject.

The bone of a subject can be imaged periodically over the course of a therapeutic treatment, and a practitioner can then compare the images and/or otherwise quantify lesion or cancer growth on the bone to determine therapeutic efficacy (e.g., if there is a differential killing effect of the cancer cells over the course of the therapeutic treatment, or a relative increase in lesion size or cancer growth on the bone). Accordingly, a method is provided for determining a likelihood of success of a therapeutic treatment in a subject.

A method of binding a conjugate a pharmaceutically acceptable salt thereof to hydroxyapatite (e.g., in a damaged or diseased bone) in a subject (e.g., in a subject in need thereof) is also provided, the method comprising administering to the subject an effective amount of: (i) any conjugate described herein or a pharmaceutically acceptable salt thereof; or (ii) a pharmaceutical composition described herein. In certain embodiments, the conjugate or pharmaceutically acceptable salt thereof has the formula:

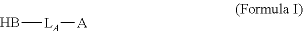

wherein HB is a radical of an (e.g., straight-chain or branched-chain) anionic, acidic, and/or electrolytic ligand (e.g., that binds hydroxyapatite); A is an active agent comprising a radio-imaging agent, a radio-sensitizing agent, a radio-protecting agent, or a radiotherapeutic agent; and L_{A} is a linker that binds an HB and A, or is absent. In certain embodiments, the conjugate or pharmaceutically acceptable salt thereof has the formula:

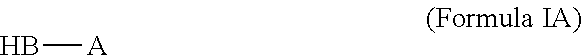

or a pharmaceutically acceptable salt thereof, wherein HB is a radical of an anionic, acidic, and/or electrolytic ligand (e.g., that binds hydroxyapatite); and A is an active agent comprising a radio-imaging agent, a radio-sensitizing agent, a radio-protecting agent, or a radiotherapeutic agent. In some embodiments, HB binds to hydroxyapatite of a damaged or diseased bone. In some embodiments, the subject has a bone disease, a bone injury, or a bone disorder (e.g., a disease, disorder, or injury described herein). In certain embodiments, the subject has a cancer (e.g., a bone cancer). In some embodiments the subject has an osteoblastic bone cancer. In some embodiments, the subject has an osteolytic bone cancer.

The conjugates and pharmaceutical compositions can be administered in unit dosage forms and/or compositions.

For methods described herein, the conjugate(s) and compositions can be administered in a single dose, or via a combination of multiple dosages, which can be administered by any suitable means, contemporaneously, simultaneously, sequentially, or separately. Where the dosages are administered in separate dosage forms, the number of dosages administered per day for each compound or composition can be the same or different. The conjugate and/or composition dosages can be administered via the same or different routes of administration. The conjugates or compositions can be administered according to simultaneous or alternating regimens, at the same or different times during the course of the therapy, concurrently in divided or single forms.

Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The conjugate/composition can be administered more than once, such as daily (1-3 or more times per day; q.d. (once a day), b.i.d. (twice a day), t.i.d. (three times a day)), weekly (including 1-3 or more times on a given day), bi-weekly (including 1-3 or more times on a given day), monthly (including 1-3 or more times on a given day), or bimonthly (including 1-3 or more times on a given day). In each case it is understood that the effective amounts described herein correspond to the instance of administration, or alternatively to the total daily, weekly, month, or quarterly dose, as determined by the dosing protocol.

### Certain Definitions

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range. The term “substantially” can allow for a degree of variability in a value or range, for example, within 90%, within 95%, 99%, 99.5%, 99.9%, 99.99%, or at least about 99.999% or more of a stated value or of a stated limit of a range.

The terms “a,” “an,” or “the” are used to include one or more than one unless the context clearly dictates otherwise. The term “or” is used to refer to a nonexclusive “or” unless otherwise indicated.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “patient” and “subject” are used interchangeably and include a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale. The patient to be treated is preferably a mammal, in particular a human being.

While the concepts of the present disclosure are illustrated and described in detail in the figures and descriptions herein, results in the figures and their description are to be considered as exemplary and not restrictive in character; it being understood that only the illustrative embodiments are shown and described and that all changes and modifications that come within the spirit of the disclosure are desired to be protected. Indeed, the numerous specific details provided are set forth to provide a thorough understanding of the present disclosure.

Any use of section headings is intended to aid reading of the document and is not to be interpreted as limiting. Further, information that is relevant to a section heading may occur within or outside of that particular section.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Various techniques and mechanisms of the present disclosure will sometimes describe a connection or link between two components. Words such as attached, linked, coupled, connected, tethered and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

### EXAMPLES

The following examples serve to illustrate the present disclosure. The examples are not intended to limit the scope of the claimed invention in any way.

### Example 1

### Animal Model

Female Balb/C mice (5˜16 weeks old) were purchased from Charles River or The Jackson Laboratory. The subjects received 4T1 (murine breast cancer cells) transfected with luciferase (4T1/L) in their femurs or tibias. First, the skin at the joint between the femur and tibia was incised open. Next, the tendon that connects the femur and tibia was pushed aside using a 26 G needle, and the same needle was used to drill a hole at the top of the tibia or femur (the location of the growth plate). The 26 G needle was removed, and a 27 G needle attached to a syringe was inserted into the hole created by the 26 G needle to inject 1,000˜100,000 4T1/L cells into the bone. Finally, the incision site was sutured closed.

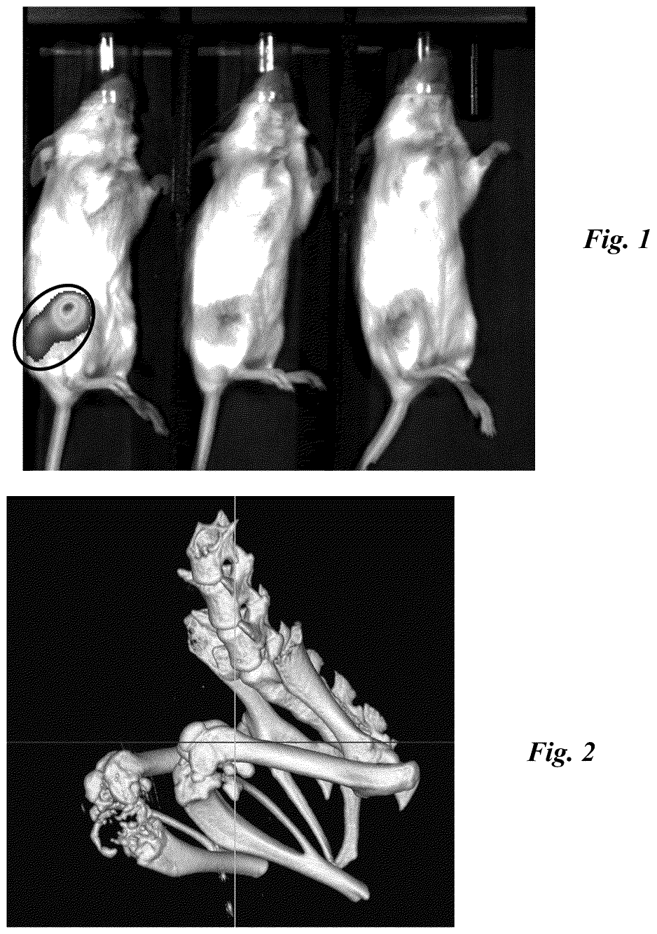

Three days after the initial tumor challenge, the mice received an intraperitoneal injection of 100 μL of luciferin dissolved in PBS (15 mg/mL). Fifteen minutes after the injection, the mice were anesthetized and were placed in an AMI small animal luminescence and fluorescence imaging instrument to measure and observe luciferin bioluminescence. The luciferin bioluminescence allowed visual confirmation of tumor development and progress. FIG. 1 shows luciferin bioluminescence of the tumor growing in the right femur of the first mouse two weeks after initial tumor challenge.

After confirming tumor development, mice were imaged using microCT to follow bone degradation. The mice were anesthetized and then placed on the bed of the microCT. Then, both their right and left tibias and femurs were scanned, and the image was reconstructed to provide a 3D image of their bones. FIG. 2 shows a microCT image of a mouse two weeks after initial tumor challenge in the right tibia, evidencing severe bone resorption due to tumor growth.

### Example 2

### Fluorescence Imaging Conjugate Synthesis

On 2-chlorotrityl resin, D-aspartic acid was loaded by reacting 1 eq of resin with 5 eq of Fmoc-D-Asp(t-butyl)-OH and 15 eq of N,N-Diisopropylethylamine (DIPEA) in dichloromethane (DCM) for 4 hours. Next, the resin was capped by reacting the resin with 17:2:1 DCM:MeOH:DIPEA for 15 minutes three times. After the loading, the resin was submitted to an AAPPTEC automatic peptide synthesizer to couple 9 D-aspartic acids and 1 cysteine, resulting in the synthesis of a 10 D-aspartic acid and 1 cysteine peptide (DD10-C). Briefly, the peptide was synthesized by deprotecting Fmoc on amine of last amino acid attached to the resin, pre-activating consequent amino acid with 5 eq of PyBop with 15 eq of NMM or HBTU and reacting the resin with preactivated amino acid for 40 minutes. Each amino acid coupling was repeated twice to ensure maximum yield of the final peptide. The Fmoc protecting group on amine of terminal cysteine was not deprotected to serve as an albumin binder as well as a UV-active group to facilitate later purification of the conjugate. After the completion of the synthesis, DD10-C was deprotected and cleaved from the resin by reacting the DD10-C attached resin with 95% TFA, 2.5% TIPS, 2.5% distilled H₂O, and 10 mM TCEP at room temperature (RT) for 2 hours. The peptide was precipitated out by saturating the collected cleavage solution with diethyl ether. The precipitate was purified using HPLC with the method of 5-50% ammonium acetate buffer (pH=5, 20 mM) to acetonitrile.

A near infrared dye 50456 conjugate was prepared by first reacting 1 eq of N-Boc-tyramine with 1 eq of 50456 and 5 eq of CsCO₃ in DMSO at RT for 4 hours. The conjugate was precipitated out by saturating the reaction with ethyl acetate and, Boc protecting group was deprotected by reacting N-Boc-tyramine-S0456 with 40% of TFA in DCM for 40 minutes. The conjugate was purified using HPLC with the method of 5-60% ammonium acetate buffer (pH=7, 20 mM) to acetonitrile. Purified tyramine-S0456 was conjugated to 3 maleimidopropionic acid (3MPA) by reacting 1 eq of tyramine-S0456 to 1 eq of 3MPA preactivated with 1 eq of PyBOP and 5 eq of DIPEA. The reaction was achieved at RT for 4 hours. Final 3MPA-tyramine-S0456 was purified and collected using HPLC method of 5-60% ammonium acetate buffer (pH=7, 20 mM) to acetonitrile.

Finally, 3MPA-tyramine-S0456 was coupled to DD10-Cys by reacting 1 eq of each reactant in anhydrous DMSO at RT for 4 hours. The final fluorescent S0456-C-DD10 was purified and collected using HPLC method of 5-50% ammonium acetate buffer (pH=7, 20 mM) to acetonitrile (FIG. 4).

### Example 3

### Fluorescence Imaging

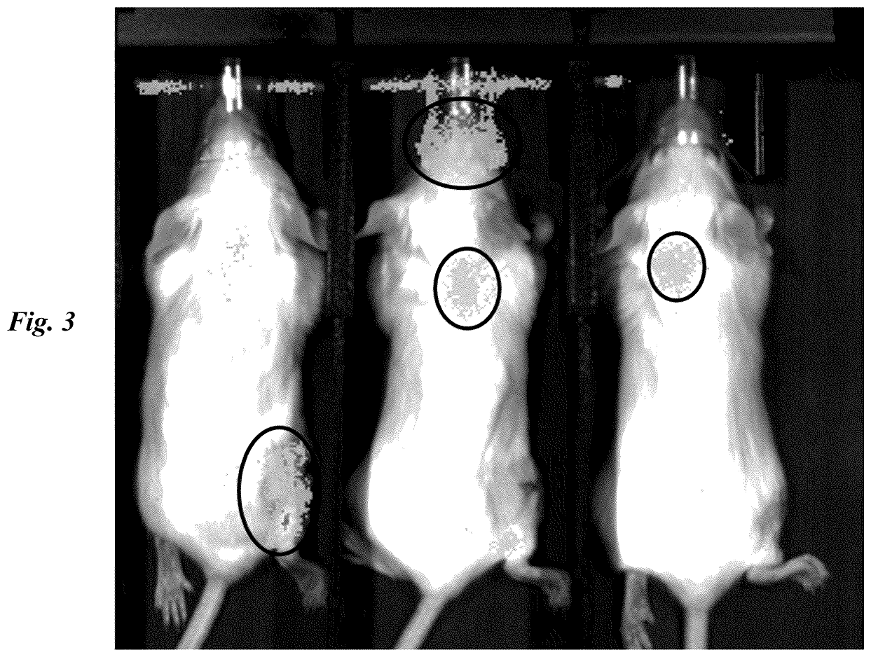

After confirmation of both tumor and bone lesion development, the targetability of hydroxyapatite binding moiety DD10 (or a poly-aspartic peptide) was tested. The subjects in Example 1 received subcutaneous injection of S0456-C-DD10 (4 nmol/100 μL). Twenty-four hours after the injection, the mice were anesthetized and were placed in an AMI small animal luminescence and fluorescence imaging instrument to visualize localization of the fluorescent conjugate. FIG. 3 shows fluorescence of bone-targeting conjugate in the right femur of the first mouse two weeks after initial tumor challenge.

### Example 4

### Radio-Imaging and Radiotherapeutic Conjugate Synthesis

On 2-chlorotrityl resin, lysine was loaded by reacting 1 eq of resin with 5 eq of Fmoc-Lys(Alloc)-OH and 15 eq of DIPEA in DCM for 4 hours. Next, the resin was capped by reacting the resin with 17:2:1 DCM:MeOH:DIPEA for 15 minutes three times. After the loading, the resin was submitted to an AAPTEC automatic peptide synthesizer to couple 1 phenylalanine and 10 D-aspartic acids onto lysine. Briefly, the peptide was synthesized by deprotecting Fmoc on the amine of the last amino acid attached to the resin, pre-activating the consequent amino acid with 5 eq of PyBop with 15 eq of NMM or HBTU and reacting the resin with preactivated amino acid for 40 minutes. Each amino acid coupling was repeated twice to ensure maximum yield of the final peptide. At the end of the synthesis peptides K(Alloc)-F-DD10 and K(Alloc)-V-M-F-DD10 were obtained.

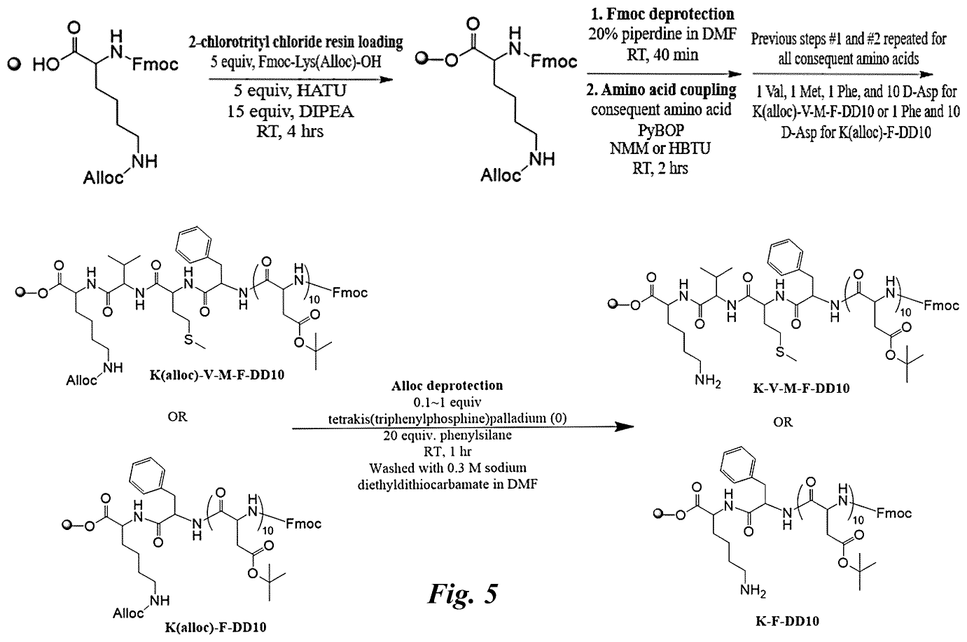

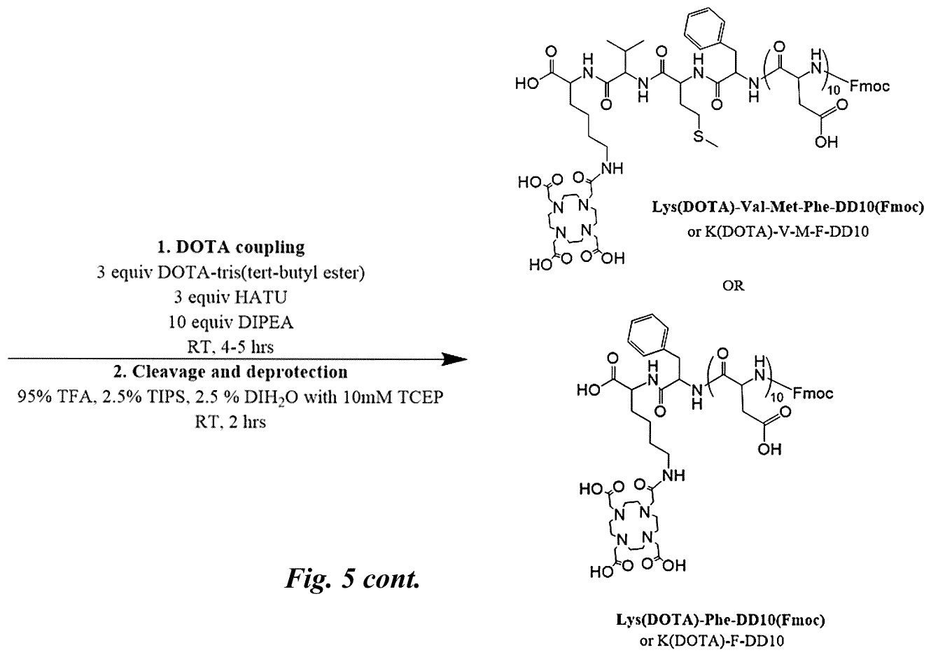

After the completion of the synthesis, alloc was removed from Lys by reacting the resin with 0.1-1 eq of tetrakis(triphenylphosphine)palladium (0) and 20 eq of phenylsilane in DCM. The deprotection lasted 40 minutes and was repeated twice to obtain K—F-DD10 and K—V-M-F-DD10. Next, the resin was washed with dimethylformamide (DMF) 3 times, sodium diethyldithiocarbamate solution (0.03 M, in DMF) three times, and DCM three times to ensure removal of the palladium. Then 3 eq of DOTA-tris(t-butyl ester) preactivated with 3 eq of HATU and 10 eq of DIPEA was reacted with the resin. The Fmoc protecting group on the amine of terminal D-aspartic acid was not deprotected to serve as an albumin binder as well as a UV-active group to facilitate purification. Both peptides were deprotected and cleaved from the resin using 95% TFA, 2.5% TIPS, 2.5% distilled H₂O with 10 mM TCEP at RT for 2 hours. The collected cleavage solution was saturated with diethyl ether to precipitate out the peptides, and the products were purified using HPLC with 5-55% ammonium acetate buffer (pH=5, 20 mM) to acetonitrile to yield K(DOTA)-F-DD10 and K(DOTA)-V-M-F-DD10 (FIGS. 5-7).

### Example 5

### In-111 Chelation to Radio-Imaging Conjugates

The radioisotope ¹¹¹In (100˜300 μCi) was chelated to the DOTA of K(DOTA)-F-DD10 (FIG. 6) and K(DOTA)-V-M-DD10 (FIG. 7). FIG. 18 shows conjugate K(DOTA)-F-DD10 chelated to ¹¹¹In. First, stock solutions for both conjugates were prepared in ultrapure sodium acetate buffer (pH 5, 1 M) at a concentration of 10 mM. Then the conjugates were reacted with 111-In so the chelation concentration was 100˜300 μCi/10 nmol, and the reaction concentration was at least 1 mM. The mixture was reacted at 90° C. for 15 minutes, and the efficiency of the chelation was checked by submitting diluted sample of the mixture into radioHPLC with the method of 5-95% ultrapure ammonium acetate buffer (pH=5, 20 mM) to ultrapure acetonitrile. Next, 5 mM of DTPA were added so the final concentration was 0.2 mM. DTPA is a chelator that will remove any unchelated, free radioisotopes. Finally, the mixture was diluted with 1% DMSO in PBS with 100 mg/mL of ascorbate and 5 mg/mL of methionine so the final concentration of the conjugate was 10 nmol/100 μL. Both ascorbic acid and methionine are radio-stabilizers, which will assist in preventing conjugates from degrading due to radioactive isotopes.

### Example 6

### Radio-Imaging

Female Balb/C mice (5 to 8 weeks old) with intratibial 4T1/L murine breast tumors received single tail-vein injections of ¹¹¹In chelated K(DOTA)-F-DD10 (or K(DOTA)-V-M-DD10 at the dosage of 100˜300 μCi/10 nmol. A single photon emission computer tomography (SPECT)/computed tomography (CT) instrument was used to visualize the biodistribution of the radio-chelated conjugates at 1, 4, 12, 24, 48, 72, 96, and 120 hours post-injection. Their body weights were measured daily. After a week of initial radio-imaging agent injection, the mice were imaged for luciferin bioluminescence every other day to follow tumor growth and/or reduction. Additionally, the mice were scanned with a microCT once or twice a week to monitor bone degradation and/or formation. After the study the mice were euthanized, and their organs were collected and fixed for histology or any other further analysis.

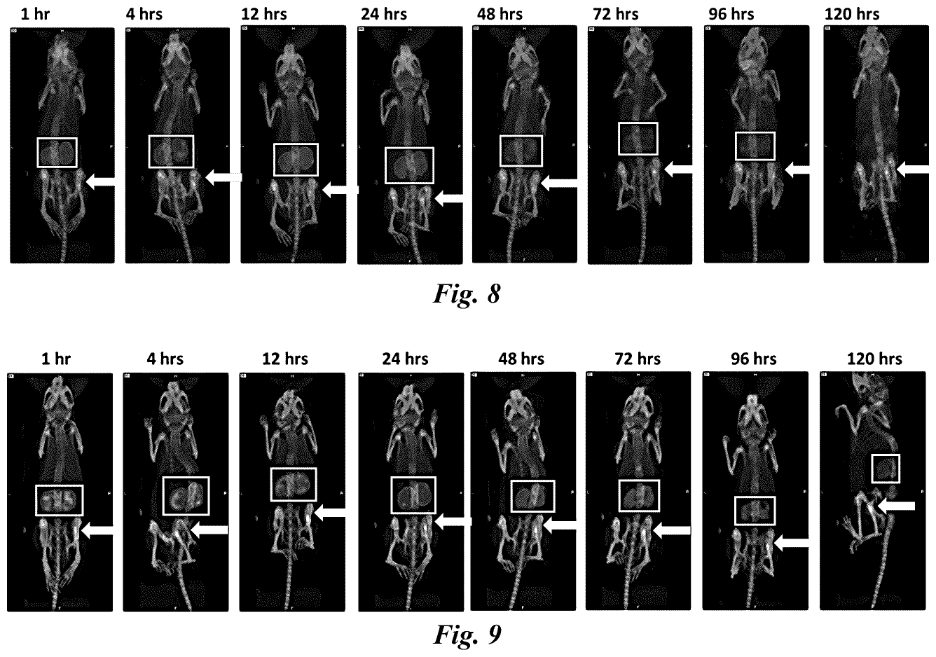

FIG. 8 shows the SPECT/CT images of tibial tumor-bearing mice injected with In-111 chelated K(DOTA)-F-DD10 (220 μCi of 200 μCi/10 nmol dosage injected). The arrows indicate the site of tumor growth, and the white boxes indicate kidney.

FIG. 9 shows the SPECT/CT images of tibial tumor bearing mouse injected with In-111 chelated K(DOTA)-V-M-F-DD10 (375 μCi of 300 μCi/10 nmol dosage injected). The arrows indicate the site of tumor growth, and the white boxes indicate kidney.

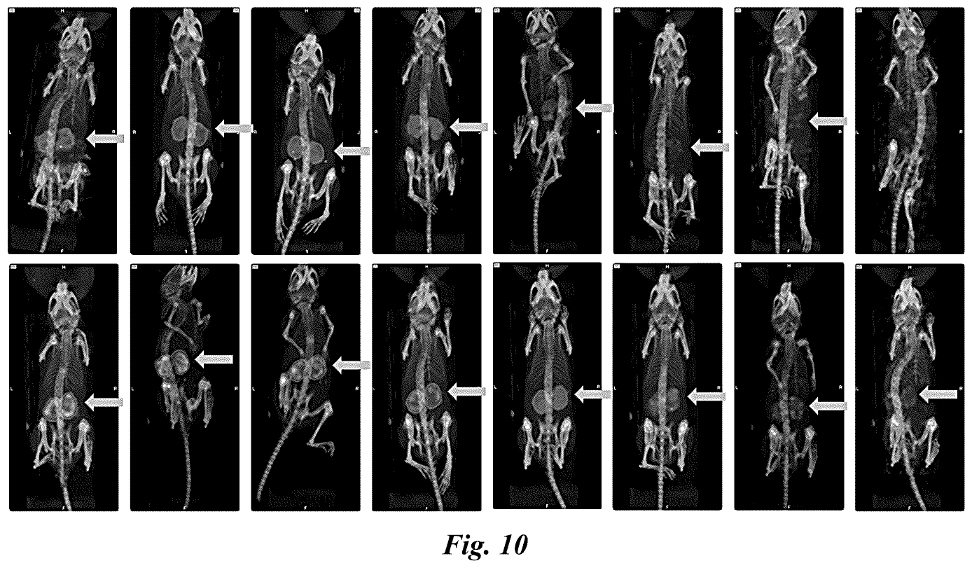

FIG. 10 shows SPECT/CT images of healthy mice injected with ¹¹¹In chelated K(DOTA)-F-DD10 (160 μCi of 200 μCi/10) or ¹¹¹In chelated K(DOTA)-V-M-F-DD10 (350 μCi of 300 μCi/10 nmol dosage injected). The white boxes indicate kidney.

In all images, offsite localization to joints (especially at shoulders and between femur and tibia) were visualized due to age of the subjects. Mice at ages 4˜12 weeks are considered immature, and at this age their growth plates are not completely closed, exposing hydroxyapatites as bones and joints are rapidly and excessively being built.

### Example 7

### Lu-177 Chelation to Radiotherapeutic Conjugates

For chelation, 1˜6 mCi of the radioisotope ¹⁷⁷Lu was chelated to the DOTA of K(DOTA)-F-DD10 (FIG. 6). FIG. 17 shows conjugate K(DOTA)-F-DD10 chelated to ¹⁷⁷Lu. First, stock solution of the conjugate was prepared in ultrapure sodium acetate buffer (pH 5, 1 M) at a concentration of 10 mM. Then the conjugates were reacted with 177-Lu so the chelation concentration was 1˜6 mCi/10 nmol and the reaction concentration was at least 1 mM. The mixture was reacted at 90° C. for 15 minutes, and the efficiency of the chelation was checked by submitting diluted sample of the mixture into radioHPLC with the method of 5-95% ultrapure ammonium acetate buffer (pH=5, 20 mM) to ultrapure acetonitrile. Next, 5 mM of DTPA were added so the final concentration was 0.2 mM. DTPA is a chelator that will remove any unchelated, free radioisotopes. Finally, the mixture was diluted with 1% DMSO in PBS with 100 mg/mL of ascorbate and 5 mg/mL of methionine so the final concentration of the conjugate was 10 nmol/100 μL. Both ascorbic acid and methionine are radio-stabilizers, which will assist in preventing conjugates from degrading due to radioactive isotopes.

### Example 8

### Radiotherapy

Female Balb/C mice (14 to 16 weeks old) with intratibial 4T1/L murine breast tumors received single tail-vein injections of Lu-111 chelated K(DOTA)-F-DD10 or K(DOTA)-V-M-DD10 at the dosage of 1˜6 mCi/10 nmol. A SPECT/CT instrument was used to visualize the biodistribution of the radio-chelated conjugates at 2 to 240 hours post-injection. Their body weights were measured daily. After a week of initial radio-imaging agent injection, the mice were imaged for luciferin bioluminescence every other day to follow tumor growth and/or reduction. Additionally, the mice were scanned with a microCT once or twice a week to monitor bone degradation and/or formation. After the study the mice were euthanized, and their organs were collected and fixed for histology or any other further analysis.

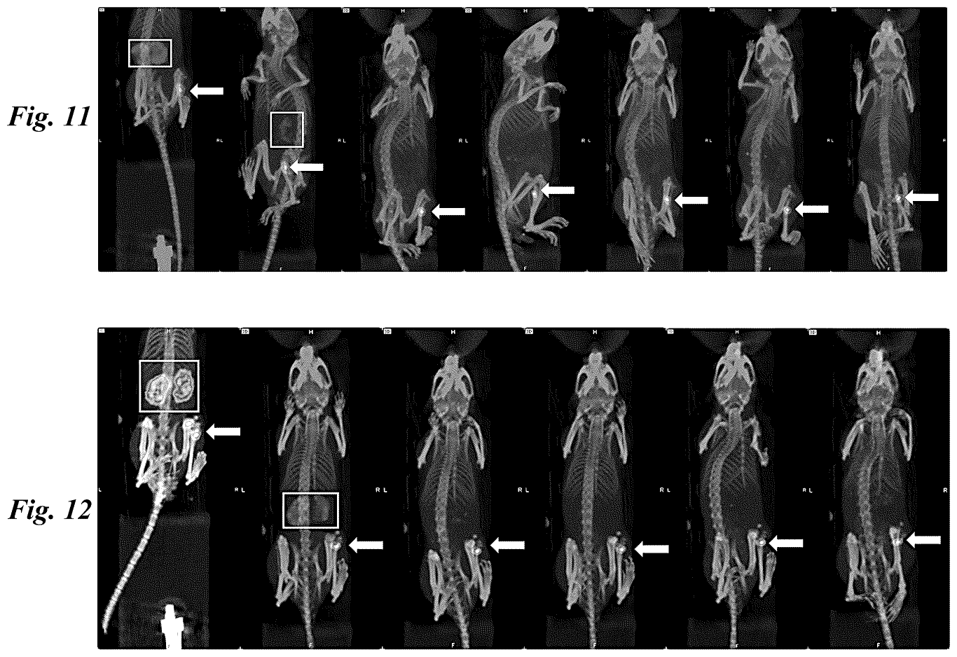

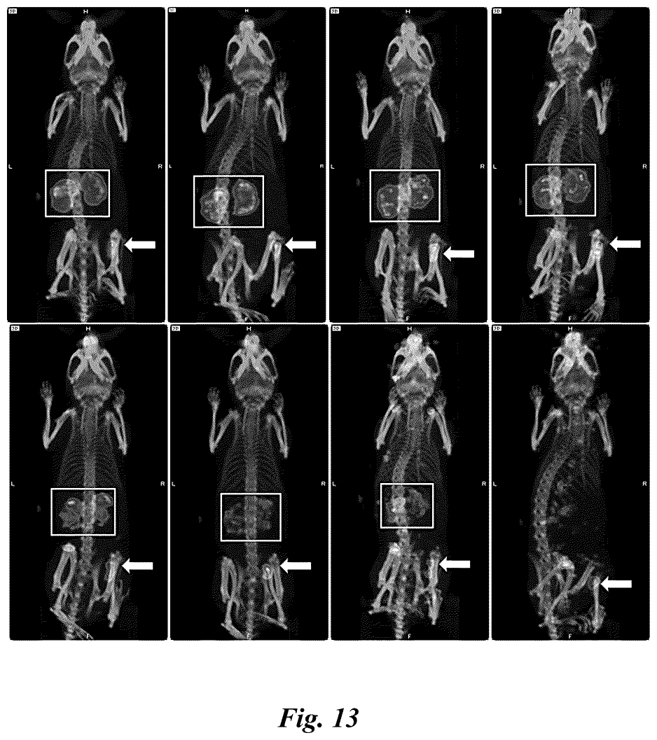

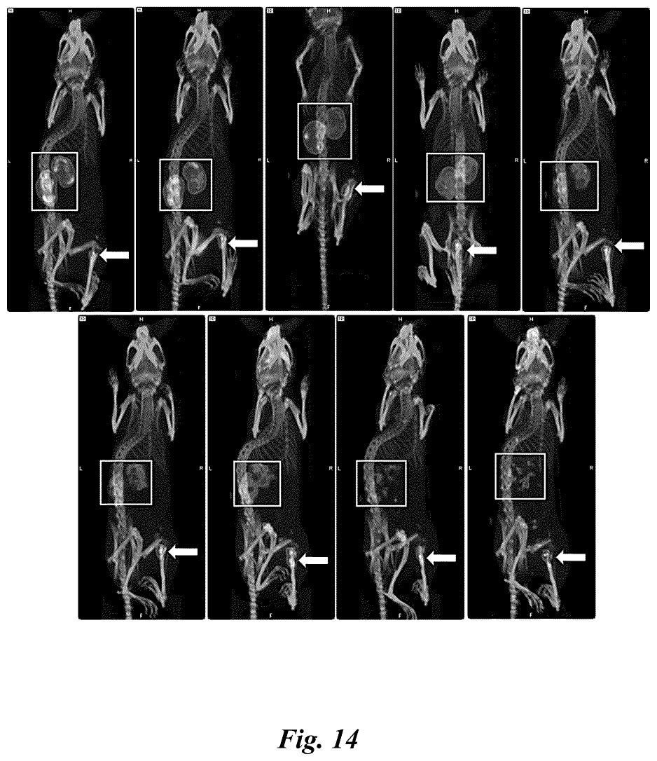

FIG. 11 shows the SPECT/CT images of a tibial tumor bearing mouse injected with Lu-177 chelated K(DOTA)-F-DD10 (0.962 mCi of 1.5 mCi/10 nmol dosage injected). The arrows indicate the site of tumor growth, and the white boxes indicate kidney. From left to right: imaged at 3 hours, 24 hours, 48 hours, 72 hours, 96 hours, 120 hours, and 168 hours post radiotherapeutic agent injection. FIG. 12 shows the SPECT/CT images of a tibial tumor bearing mouse injected with Lu-177 chelated K(DOTA)-F-DD10 (3.0 mCi of 3.0 mCi/10 nmol dosage injected). The arrows indicate the site of tumor growth, and the white boxes indicate kidney. From left to right: imaged at 3 hours, 24 hours, 72 hours, 96 hours, 120 hours, and 168 hours post radiotherapeutic agent injection. FIG. 13 shows the SPECT/CT images of a tibial tumor bearing mouse injected with Lu-177 chelated K(DOTA)-F-DD10 (3.57 mCi of 3.0 mCi/10 nmol dosage injected). The arrows indicate the site of tumor growth, and the white boxes indicates kidney. Top row from left to right: imaged at 2 hours, 12 hours, 24 hours, and 48 hours post radiotherapeutic agent injection. Bottom row from left to right: imaged at 72 hours, 96 hours, 144 hours, and 196 hours post radiotherapeutic injection. FIG. 14 shows the SPECT/CT images of a tibial tumor bearing mouse injected with Lu-177 chelated K(DOTA)-F-DD10 (5.50 mCi of 6.0 mCi/10 nmol) dosage injected). The arrows indicate the site of tumor growth, and the white boxes indicate kidney. Top row from left to right: imaged at 2 hours, 12 hours, 48 hours, 48 hours, and 72 hours post radiotherapeutic agent injection. Bottom row from left to right: imaged at 96 hours, 144 hours, 196 hours, and 240 hours post radiotherapeutic agent injection.

In all images, minimal offsite localization was observed as mice are at least 12 weeks and older. At this age, mice are considered as skeletally mature, and their growth plates are mostly or completely closed.

All polyaspartic acid conjugates, S0456-C-DD10, K(DOTA)-F-DD10 and K(DOTA)-V-M-F-DD10, demonstrated specific localization to tumor-induced bone lesions. The conjugates adhered to the bone lesions for at least 7 days post-injection. Offsite localization was observed in the kidneys but both conjugates cleared out quickly. The majority of KDFDD10 was removed from the kidney within 24-48 hours, whereas the majority of KDVMFDD10 was removed within 48-72 hours. KDFDD10 showed faster renal clearance than KDVMFDD10. Offsite localization was observed in the joints but this was as expected since mice were young (i.e., 5-6 weeks old), and their growth plates were still in development. This problem was resolved by using older mice (i.e., 12 weeks and older).

### Example 9

### Novel Radio-Imaging and Radiotherapeutic Conjugate Synthesis

To test if a brush border membrane (BBM) peptidase linker (quick-release) expedites the renal clearance, a bone-targeting radio-imaging and radiotherapeutic conjugate with BBM peptidase linker was incorporated to link a bone targeting acidic peptide to a chelator.

On 2-chlorotrityl chloride resin, lysine was loaded by reacting 1 eq of resin with 5 eq of Fmoc-Lys(Alloc)-OH and 15 eq of DIPEA in DCM for 4 hours. Next, the resin was capped by reacting the resin with 17:2:1 DCM:MeOH:DIPEA for 15 minutes, three times. After the loading, the resin was submitted to an AAPTEC automatic peptide synthesizer to couple Val, Met, and a chelator such as DOTA.

Briefly, the peptide was synthesized by deprotecting Fmoc on the amine of the last amino acid attached to the resin, pre-activating the consequent amino acid with 5 eq of PyBop with 15 eq of NMM or HBTU, and reacting the resin with preactivated amino acid for 40 minutes. Each amino acid coupling was repeated twice to ensure maximum yield of the final peptide. At the end of the synthesis, K(Alloc)-Val-Met-DOTA was obtained.

On 2-chlorotrityl chloride resin, D-aspartic acid was loaded by reacting 1 eq of resin with 5 eq of Fmoc-D-Asp(OtBu)-OH and 15 eq of DIPEA in DCM for 4 hours. Next, the resin was capped by reacting the resin with 17:2:1 DCM:MeOH:DIPEA for 15 minutes, three times. After the loading, the resin was submitted to an AAPTEC automatic peptide synthesizer to couple 9 D-Asp and 1 or more aromatic amino acids such as Phe. Briefly, the peptide was synthesized by deprotecting Fmoc on the amine of the last amino acid attached to the resin, pre-activating the consequent amino acid with 5 eq of PyBop with 15 eq of NMM or HBTU, and reacting the resin with preactivated amino acid for 40 minutes. Each amino acid coupling was repeated twice to ensure maximum yield of the final peptide. The peptide was cleaved in protected form by reacting the resin with 1:1:8 acetic acid:TFE:DCM for 30 minutes. At the end of the synthesis, D-Asp₁₀(OtBu)-Phe₂-Fmoc was obtained.

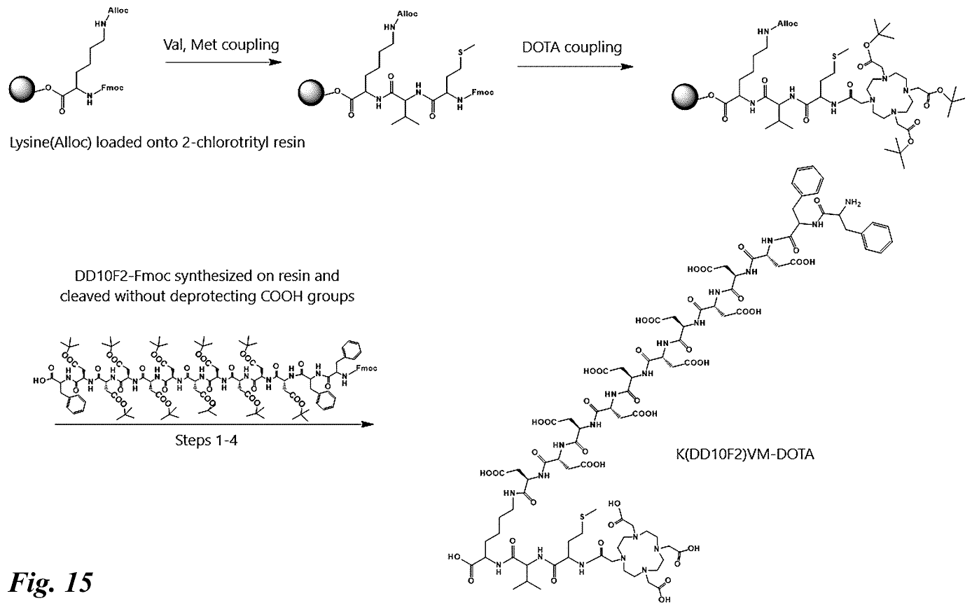

After the completion of the synthesis, alloc was removed from Lys by reacting the resin with 0.1-1 eq of tetrakis(triphenylphosphine)palladium (0) and 20 eq of phenylsilane in DCM. The deprotection lasted 40 minutes and was repeated twice to obtain K—V-M-DOTA. Next, the resin was washed with DMF 3 times, sodium diethyldithiocarbamate solution (0.03 M in DMF) three times, and DCM three times to ensure removal of the palladium. Then 1 eq of protected D-Asp₁₀(OtBu)-Phe₂-Fmoc peptide preactivated with 3 eq of HATU and 10 eq of DIPEA was reacted with the resin. The Fmoc protecting group on the amine of terminal Phe was deprotected. The peptide was deprotected and cleaved from the resin using 95% TFA, 2.5% TIPS, 2.5% distilled H₂O with 10 mM TCEP at RT for 2 hours. The collected cleavage solution was saturated with diethyl ether to precipitate out the peptides, and the products were purified using HPLC with 5-55% ammonium acetate buffer (pH=5, 20 mM) to acetonitrile to yield K(DD₁₀F₂)VM-DOTA (FIG. 15). FIG. 16 shows structure of the conjugate.

### Example 10

### Synthesis of Bone Targeted D-Gla Peptides Conjugated to DOTA

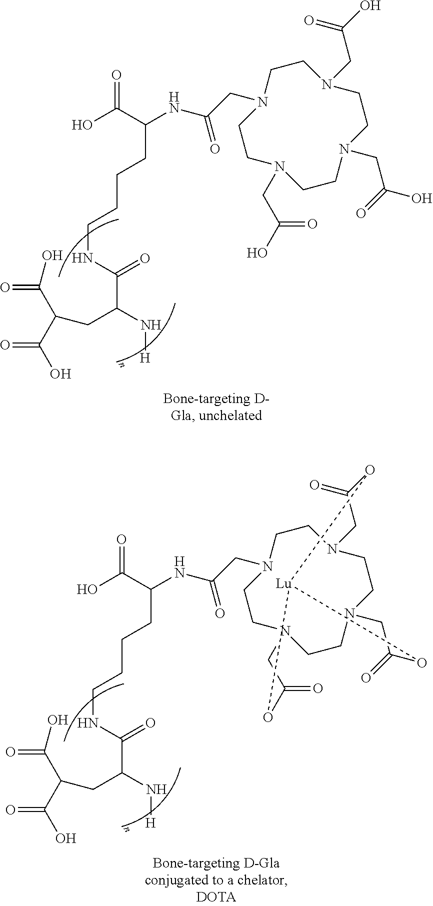

D-Gla bone targeting peptides were prepared according to the following protocol. Fmoc-Lys(Alloc)-OH was initially loaded onto solid phase under standard peptide coupling conditions. Following successful attachment, the Fmoc protecting group was selectively removed for subsequent conjugation. A DOTA-based chelator was then coupled to the deprotected lysine residue, enabling site-specific incorporation of the metal-binding moiety. Following DOTA conjugation, the Alloc protecting group of the lysine was removed. This orthogonal deprotection strategy preserved the integrity of the DOTA moiety while liberating the side-chain amine for further functionalization. n acidic amino acids such as Fmoc-DGla(OtBu)-OH, where then conjugated to the liberated side-chain amine. After coupling with the last amino acid, the Fmoc protecting group was removed under standard conditions, and the fully assembled conjugate was cleaved from the solid support using a TFA-based cleavage solution.

FIG. 25 shows synthesis schemes of this protocol.

### Example 11

### Comparison of Organ Uptake

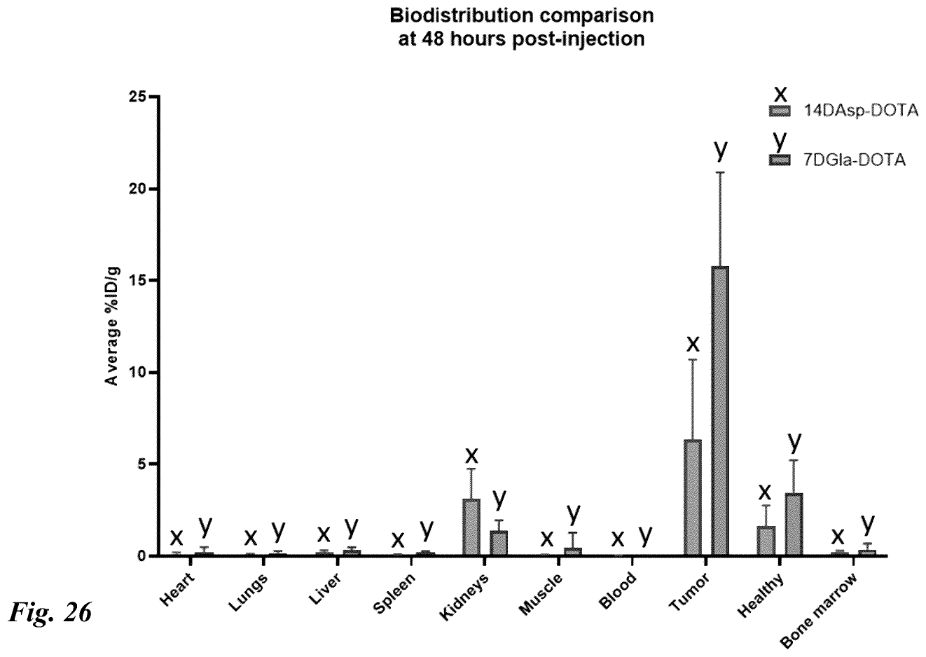

Intratibial 4T1 murine breast cancer tumor-bearing mice were administered equivalent doses of acidic peptide-DOTA conjugates having either 14 D-aspartic acid residues (D-Asp conjugate) or 7 D-γ-carboxyglutamic acid residues (D-Gla conjugate). At 48 hours post-injection, the D-Gla conjugate exhibited enhanced tumor uptake and reduced kidney uptake relative to the D-Asp conjugate (FIG. 26).

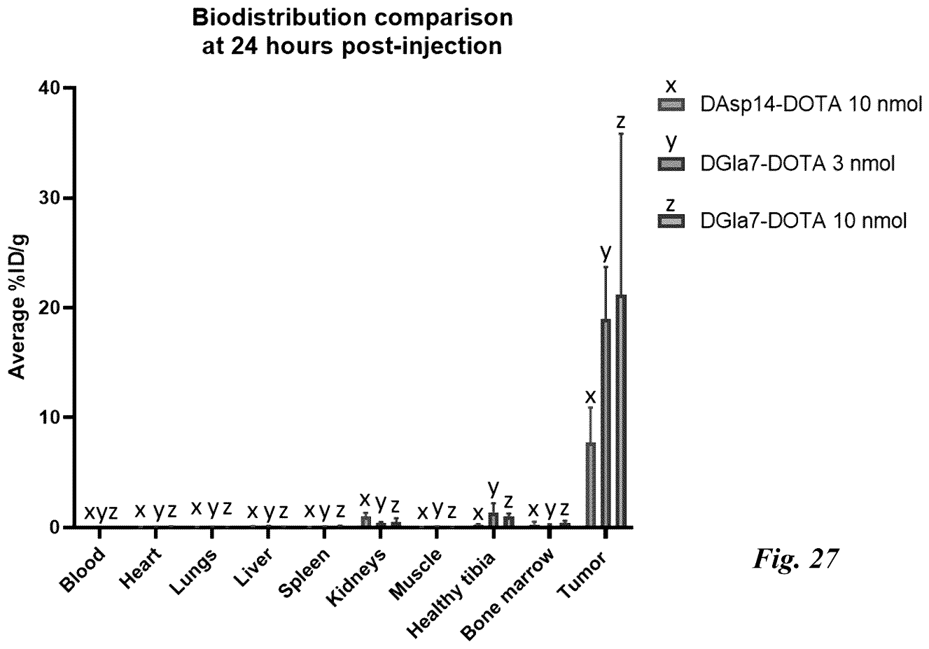

Intratibial C4-2 human prostate cancer tumor-bearing mice were administered different dosages (3 nmol or 10 nmol) of acidic peptide-DOTA—either the D-Asp conjugate or the D-Gla conjugate. At 24 hours post-injection, administration of a lower dose of the D-Gla conjugate (3 nmol) resulted in greater tumor uptake and reduced kidney uptake compared to a higher dosage of the D-Asp conjugate (10 nmol) (FIG. 27).

1. A conjugate of Formula I:
HB-L_{A}-A (Formula I)
or a pharmaceutically acceptable salt thereof, wherein:
HB comprises a radical of a D-γ-carboxyglutamic acid ligand, L-γ-carboxyglutamic acid ligand, or a mixture of the foregoing or derivatives thereof that binds hydroxyapatite;
A is an active agent comprising a radio-imaging agent, a radio-sensitizing agent, a radio-protecting agent, or a radiotherapeutic agent; and
L_{A} is a linker that binds HB and A, or absent.

2. The conjugate of claim 1, wherein HB is a straight chain or branched D-γ-carboxyglutamic acid ligand, L-γ-carboxyglutamic acid ligand, or a mixture of the foregoing.

3. The conjugate of claim 1, wherein HB comprises an amino acid or a derivative thereof, and least a third of the amino acid sequence of HB is acidic in nature.

4. The conjugate of claim 3, wherein HB comprises 1 to 100 D-γ-carboxyglutamic acid residues, L-γ-carboxyglutamic acid residues, or a mixture of the foregoing or derivatives thereof.

5. The conjugate of claim 4, wherein HB comprises 7-20 D-γ-carboxyglutamic acid residues.

6. The conjugate of claim 1, wherein HB further comprises (i) a serum albumin binder (AB) conjugated directly to HB, or (ii) AB and a linker L_{AB} (AB-L_{AB}), wherein AB-L_{AB} is conjugated to HB via L_{AB}.

7. The conjugate of claim 6, wherein L_{A}, L_{AB}, or both L_{A} and L_{AB} is/are in an L-configuration or a D-configuration.

8. The conjugate of claim 1, wherein one or more peptide bonds of HB and/or A are arranged in a relative cis orientation or a relative trans orientation.

9. The conjugate of claim 1, wherein either or both of L_{A} and L_{AB} comprise(s) a slow-release linker.

10. The conjugate of claim 1, wherein L_{A} comprises a quick-release linker.

11. The conjugate of claim 1, wherein L_{A} comprises one or more amino acids and/or a brush border membrane (BBM) linker.

12. The conjugate of claim 1, wherein L_{A} comprises a BBM linker comprising Met-Val-Lys or Gly-Tyr-Lys.

13. The conjugate of claim 1, wherein L_{A} comprises L₁ or L₂: 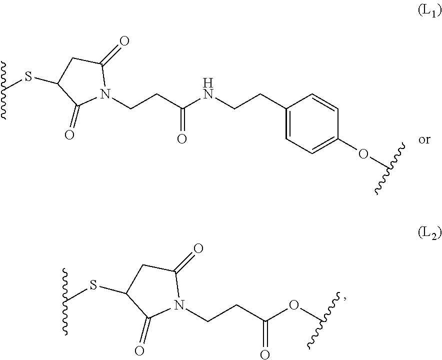 [SMILES: *Oc1ccc(CCNC(=O)CCN2C(=O)CC(S*)C2=O)cc1 ; "or" ; *OC(=O)CCN1C(=O)CC(S*)C1=O]
in which 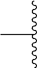
represents the point of attachment between L_{A} and A or L_{A} and HB.

14. The conjugate of claim 1, wherein either or both of L_{A} and L_{AB} comprise(s) a spacer.

15. The conjugate of claim 1, wherein A comprises a chelator.

16. The conjugate of claim 1, wherein A comprises DOTA (1,4,7,10-tetraazacyclododecane-1,4,7,10-tetraacetic acid) or a derivative thereof.

17. The conjugate of claim 15, wherein the chelator chelates or binds to a radioisotope.

18. The conjugate of claim 17, wherein the radioisotope is a therapeutic radioisotope comprising an α-emitting radioisotope or a β-emitting radioisotope.

19. The conjugate of claim 16, wherein the radioisotope comprises lutetium (Lu) or indium (In).

20. The conjugate of claim 19, wherein the β-emitting radioisotope is ¹⁷⁷Lu.

21. The conjugate of claim 18, wherein the chelator is an imaging radioisotope.

22. The conjugate of claim 21, wherein the imaging radioisotope is a positron emission tomography (PET)-imaging radioisotope or a T-emitting radioisotope that is ¹¹¹In.

23. The conjugate of claim 1 having one of the following structures:

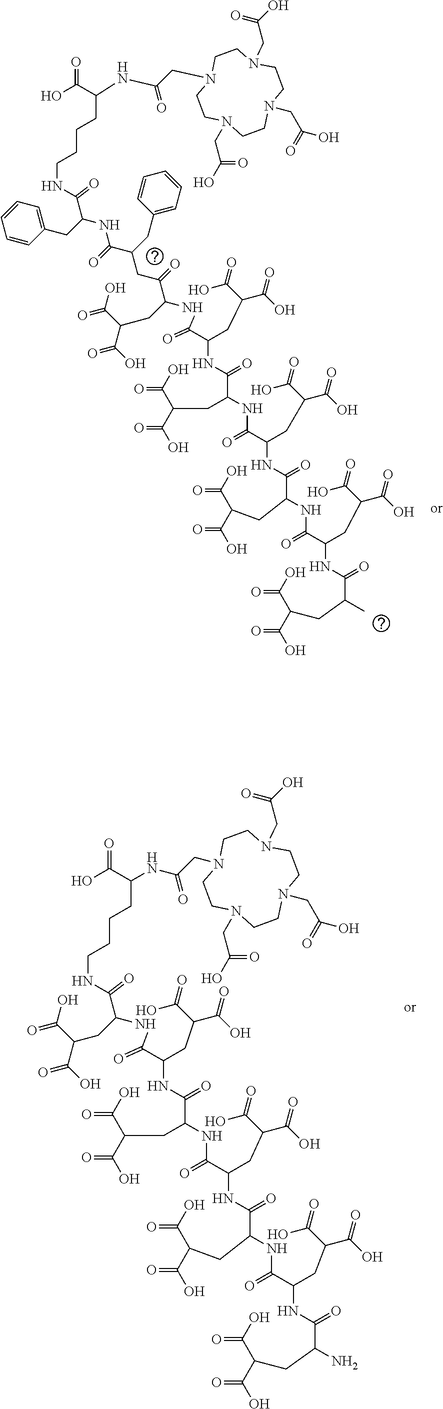

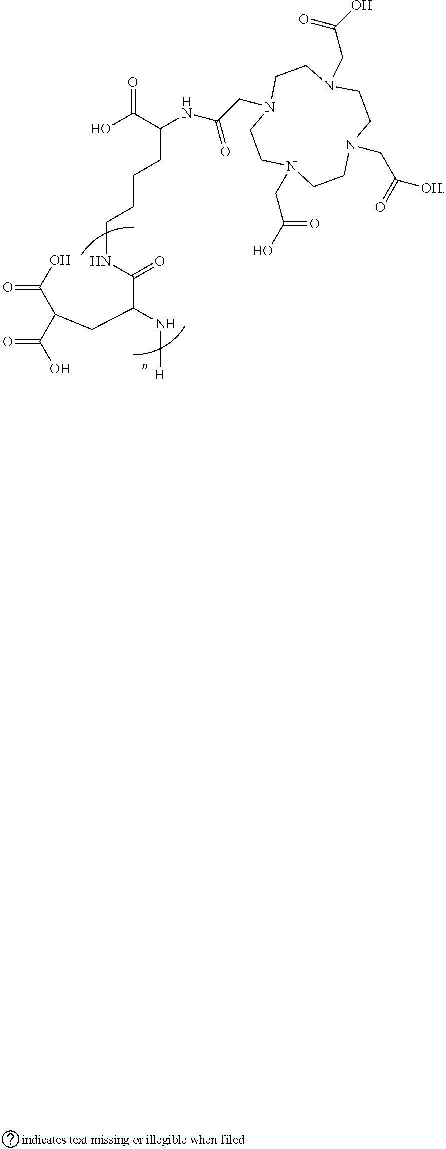

24. A pharmaceutical composition comprising a conjugate of Formula I: 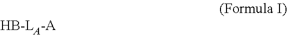
or a pharmaceutically acceptable salt thereof, wherein:
HB comprises a radical of a D-γ-carboxyglutamic acid ligand, L-γ-carboxyglutamic acid ligand, or a mixture of the foregoing or derivatives thereof that binds hydroxyapatite,
A is an active agent comprising a radio-imaging agent, a radio-sensitizing agent, a radio-protecting agent, or a radiotherapeutic agent, and
L_{A} is a linker that binds HB and A, or absent; and
a pharmaceutically acceptable carrier or excipient.

25. The pharmaceutical composition of claim 24, which further comprises a radiosensitizer, a radioprotector, an immunotherapeutic agent, a chemotherapeutic agent, an anti-cancer agent, and/or a hormone therapeutic agent.

26. A method of imaging and/or treating a bone in a subject, which method comprises administering to the subject an effective amount of:
(i) a conjugate of Formula I: 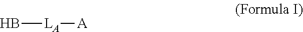
or a pharmaceutically acceptable salt thereof, wherein:
HB comprises a radical of a D-γ-carboxyglutamic acid ligand, L-γ-carboxyglutamic acid ligand, or a mixture of the foregoing or derivatives thereof that binds hydroxyapatite,
A is an active agent comprising a radio-imaging agent, a radio-sensitizing agent, a radio-protecting agent, or a radiotherapeutic agent, and
L_{A} is a linker that binds HB and A, or absent;
(ii) a first pharmaceutical composition comprising the conjugate of Formula I and a pharmaceutically acceptable carrier or excipient; or
(iii) (a) the first pharmaceutical composition and simultaneously or sequentially, in either order, (b) (i′) an active agent, or (ii′) a second pharmaceutical composition comprising the active agent and a second pharmaceutically acceptable carrier or second excipient.

27. The method of claim 26, wherein the active agent of (b) comprises a radiosensitizer, radioprotector, radioprotectant, immunotherapeutic agent, chemotherapeutic agent, anti-cancer drug, or hormone therapeutic agent.

28. The method of claim 26, wherein HB comprises 7-20 D-γ-carboxyglutamic acid residues.
